(19) United States
(12) Patent Application Publication
(10) Pub. No.: US 2009/0148866 A1
(43) Pub. Date: Jun. 11, 2009

(54) Antibodies and Improved Test Sample Handling Methods for Use in Assays for Myeloperoxidase

(75) Inventors: Saul A. Datwyler, Evanston, IL (US); David J. Hawksworth, Lake Villa, IL (US); Stephen C. Hsu, Buffalo Grove, IL (US); Matthew S. Matias, Green Oaks, IL (US); David P. Pacenti, Gurnee, IL (US); Mary S. Pinkus, Chicago, IL (US); Jessie W. Shih, Lake Forest, IL (US); Bryan C. Tieman, Elmhurst, IL (US); Joan D. Tyner, Beach Park, IL (US); Robert N. Ziemann, Lindenhurst, IL (US); Lowell J. Tyner, Chicago, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/122,244

(22) Filed: May 16, 2008

Related U.S. Application Data
(63) Continuation-in-part of application No. 12/050,061, filed on Mar. 17, 2008, now abandoned.
Application No. 12/050,061 is a continuation-in-part of application No. 11/750,507, filed on May 18, 2007.

(51) Int. Cl. G01N 33/573; C12N 5/06; C12Q 1/28; C07K 16/18

(52) U.S. Cl. 435/7.4; 435/338; 530/388.26; 435/28

(57) ABSTRACT

The present disclosure relates to isolated antibodies that can be used in an assay to determine the concentration levels of myeloperoxidase (MPO) in a test sample. Additionally, the present disclosure also relates to the use of improved test sample handling methods in assays in order to preserve the original MPO levels in the test sample.

### RELATED APPLICATION INFORMATION

This application is a continuation-in-part of U.S. application Ser. No. 12/050,061 filed on Mar. 17, 2008 which is a continuation-in-part of U.S. patent application Ser. No. 11/750,507 filed on May 18, 2007, the contents of which are herein incorporated by reference.

### TECHNICAL FIELD

The present disclosure relates to isolated antibodies that can be used in an assay to determine the concentration levels of myeloperoxidase (MPO) in a test sample. The present disclosure further relates to an assay for determining the concentration levels of MPO in a test sample using one or more of the isolated antibodies of the present disclosure. Additionally, the present disclosure also relates to the use of improved test sample handling methods in assays in order to preserve the original MPO levels in the test sample. Finally, the present disclosure relates to an improved assay which employs a test sample wherein the original MPO levels in the a test sample have been preserved to allow for the accurate detection of MPO levels in said assay.

### BACKGROUND

In the field of diagnostics, assays are used for detecting analytes in biological samples. Examples of analytes that can be detected include, drugs, hormones, infectious agents, microorganisms, antibodies and the like. The identification of one or more analytes in a biological sample can be used to diagnose cancer, heart disease, etc.

A specific type of assay, namely, an immunoassay, involves a specific binding reaction between the analyte to be detected and at least one specific binding partner. The specific binding partner (which can be an antibody, antigen, etc) specifically binds to the analyte or reacts with it. The analyte and the specific binding partner form a specific binding pair complex. An example of such a specific binding pair complex is an antibody (or antibody fragment) and antigen. However, more than one analyte or more than one specific binding partner can react with each other during each reaction.

The specific binding pair complex can then be detected. Typically, at least one specific binding partner is labeled with a detectable label such as a chromogen, fluorophore, substances capable of chemi- or electrochemiluminescence, radioisotopes, haptens, enzymes labels or substances that can form another specific binding pair such as biotin and streptavidin.

As useful as immunoassays are, they are not without their problems. For example, the specificity and sensitivity of the antibodies used in such immunoassays, is very important. If one or more antibodies used in an immunoassay exhibits poor specificity or sensitivity this could lead to false positive or false negative results. One way in which to improve or increase the specificity and sensitivity of one or more antibodies is to improve the binding affinity of said antibodies for their intended target (i.e., an antigen). Antibodies having an improved binding affinity for their intended targets should exhibit increased specificity and sensitivity.

An example of an analyte that can be detected in an assay is myeloperoxidase (MPO). MPO is a marker protein used in the diagnosis of acute cardiac syndrome (ACS). MPO (donor: hydrogen peroxide, oxidoreductase, EC 1.11.1.7) is a tetrameric, heavily glycosylated, basic (pI 10) heme protein of approximately 150 kDa. It is comprised of two identical disulfide-linked protomers, each of which possesses a protoporphyrin-containing 59-64 kDa heavy subunit and a 14 kDa light subunit (See, Nauseef, W. M, et al., Blood, 67:1504-1507 (1986)). MPO is abundant in neutrophils and monocytes, accounting for 5% and 1 to 2%, respectively, of the dry weight of these cells (See, Nauseef, W. M, et al., Blood 67:1504-1507 (1986)). The heme protein is stored in primary azurophilic granules of leukocytes and secreted into both the extracellular milieu and the phagolysosomal compartment following phagocyte activation by a variety of agonists (See, Klebanoff, S. J, et al., The Neutrophil: Functions and Clinical Disorders. Amsterdam: Elsevier Scientific Publishing Co. (1978)).

Increased concentrations of plasma MPO levels determined in patient blood samples have been shown to be linked with coronary disease. Also, increased concentrations of blood MPO levels can also be used to predict risk in patients with acute coronary syndromes, including, but not limited to, heart failure (See, Tang, W. H. et al., J. Am. College Card., 49(24): 2364-2370 (2007); Tang, W. H., et al., Am. J. Card., 98:796-799 (2006); Zhang, R., et al., JAMA, 286(17):2136-2142 (2001); Meuwese, M. C., et al., J. Am. College Card., 50(2): 159-165 (2007); G. Ndrepepa, et al., Eur. J. Clin. Invest., 38:90-96 (2007)). Thus, determination of MPO levels in patient blood samples are of clinical interest for managing such patients.

Sample collection tube type and specimen handling can affect the measured amount of an analyte in many types of assays commonly used in clinical laboratories to assay blood samples, including sandwich and competitive immunoassays, clinical chemistry assays and enzymatic assays. In particular, MPO is known to be present in leukocytes as well as free in the plasma.

However, current pre-analytical patient blood sample handling does not involve any specific steps aimed at preservation of original MPO levels. Rather, current MPO assays follow the blood storage procedures used with troponin measurements, namely collecting samples as lithium heparin plasma or serum. For example, the United States Food and Drug Administration has cleared one MPO assay for clinical use, the CardioMPO™ (a trademark of PrognostiX, Inc. (Cleveland, Ohio)) Enzyme Immunoassay Reagent Kit marketed by PrognostiX. The CardioMPO™ assay is an enzyme-linked immunosorbent assay (ELISA). The CardioMPO™ package insert states that the patient blood sample “should be stored in lithium heparin collection tubes”, and that the tubes should be placed on ice or at 2 to 8° C. immediately and then stored at 2 to 8° C. until processed (See, CardioMPO™ package insert at page 7). The problem with such blood sample handling is that MPO levels may also increase if anticoagulant collection tubes (such as lithium heparin tubes) are used as part of the sample handling process. Additionally, the original MPO levels may increase in those patients being treated prior to blood draw with an anticoagulant (such as heparin or bivalirudin).

Additionally, the current blood sample handling procedures for MPO do not take into account the reality that most blood samples will be exposed to at least some time at room temperature storage. The room temperature storage may also be for an extended time. Exposure to room temperature conditions can occur at any point before the analysis. Transportation of the sample to the laboratory from the location of blood draw (such as Emergency Department or Intensive Care Unit) is typically done at room temperature and once in the lab, there can be waiting time for centrifugation. The automated blood analyzers in clinical laboratories generally do not use cold storage conditions for the samples, so the blood samples are exposed to room temperature conditions during processing. In addition, clinical laboratories in the United States have begun implementing automated systems with multiple analyzer stations linked by automated conveyor systems. These automated systems also increase the likelihood a blood sample will be exposed to room temperature storage for extended times. Last, in addition to the reality of likely exposure to room temperature conditions, current practice does not involve any tracking or monitoring of the actual exposure of a sample to be tested for MPO levels to room temperature.

Leukocyte MPO can be released into the plasma depending on how the sample is handled and the specimen tube used to collect the sample. In this regard, the inventors have determined that pre-analytical sample handling methods currently used in advance of a MPO assay allows MPO to leak out of the leukocytes during clotting of serum and preparation of lithium heparin plasma, which causes an elevation in MPO levels which can lead to inaccurate stratification of ACS patients, and thus consequently, the wrong treatment selection.

Therefore, one object of the present disclosure is to provide antibodies capable of binding to complimentary epitopes on MPO in an assay. These antibodies exhibit good binding affinity and can be used in assays, such as, for example, immunoassays. Another object of the present disclosure is to provide for use with MPO assays methods for patient test sample handling specifically intended to preserve original patient MPO levels for accurate assay. These and other objects and advantages of the invention will be apparent from the description provided herein. The methods of the present disclosure advantageously improve MPO determinations in any clinical assay by preserving original MPO levels, and/or protecting against alterations in MPO levels caused by sample handling and storage.

### SUMMARY

In one embodiment, the present disclosure relates to a murine hybridoma cell line 1-1175-509 having A.T.C.C. Accession No. PTA-8437.

In another embodiment, the present disclosure relates to an antibody made from DNA extracted from murine hybridoma cell line 1-1175-509 having A.T.C.C. Accession No. PTA-8437.

In yet another embodiment, the present disclosure relates to a monoclonal antibody produced by murine hybridoma cell line 1-1175-509 having A.T.C.C. Accession No. PTA-8437.

In a yet another embodiment, the present disclosure relates to a murine hybridoma cell line 1-2169-715 having A.T.C.C. Accession No. PTA-8438.

In yet another embodiment, the present disclosure relates to an antibody made from DNA extracted from murine hybridoma cell line 1-2169-715 having A.T.C.C. Accession No. PTA-8438.

In yet another embodiment, the present disclosure relates to a monoclonal antibody produced by murine hybridoma cell line 1-2169-715 having A.T.C.C. Accession No. PTA-8438.

In still yet another embodiment, the present disclosure relates to an immunoassay for determining the concentration of MPO in a test sample. The immunoassay comprises the steps of:

(a) contacting a first capture antibody that binds to MPO with a test sample suspected of containing MPO to form a first capture antibody-MPO complex;

(b) contacting said test sample containing the first capture antibody-MPO complex with a second antibody that binds to MPO and that has been conjugated to a detectable label to form a second capture antibody-MPO-detection complex; and

(c) determining the amount of the capture antibody-MPO-detection complexes formed in step (b) by detecting the detectable label, wherein the amount of the second complexes formed is the amount of MPO contained in the test sample,

wherein either the first capture antibody or the second antibody is an antibody produced by murine hybridoma cell line 1-1175-509 having A.T.C.C. Accession No. PTA-8437. In this immunoassay, the first capture antibody can optionally be immobilized on a solid phase to produce an immobilized antibody.

In still yet another embodiment, the present disclosure relates to an immunoassay for determining the concentration of MPO in a test sample. The immunoassay comprises the steps of:

(a) contacting a first capture antibody that binds to MPO with a test sample suspected of containing MPO to form a first capture antibody-MPO complex;

(b) contacting said test sample containing the first capture antibody-MPO complex with a second antibody that binds to MPO and that has been conjugated to a detectable label to form a second capture antibody-MPO-detection complex; and

(c) determining the amount of the capture antibody-MPO-detection complexes formed in step (b) by detecting the detectable label, wherein the amount of the second complexes formed is the amount of MPO contained in the test sample,

wherein either the first capture antibody or the second antibody is an antibody produced by murine hybridoma cell line 1-2169-715 having A.T.C.C. Accession No. PTA-8438. In this immunoassay, the first capture antibody can optionally be immobilized on a solid phase to produce an immobilized antibody.

In still yet another embodiment, the present disclosure relates to an immunoassay for determining the concentration of MPO in a test sample. The immunoassay comprising the steps of:

(a) contacting a first capture antibody that binds to MPO with a test sample suspected of containing MPO to form a first capture antibody-MPO complex, wherein the first capture antibody is an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438;

(b) contacting said test sample containing the first capture antibody-MPO complex with a second antibody that binds to MPO and that has been conjugated to a detectable label to form a second capture antibody-MPO-detection complex, wherein the second antibody is an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438, provided that the first capture antibody and the second antibody are not identical; and

(c) determining the amount of the capture antibody-MPO-detection complexes formed in step (b) by detecting the detectable label, wherein the amount of the second complexes formed is the amount of MPO contained in the test sample. In this immunoassay, the first capture antibody can optionally be immobilized on a solid phase to produce an immobilized antibody. Preferably, in this immunoassay, the first capture antibody is an antibody produced by murine hybridoma cell line 1-1175-509 having A.T.C.C. Accession No. PTA-8437 and the second antibody is an antibody produced by murine hybridoma cell line 1-2169-715 having A.T.C.C. Accession No. PTA-8438.

In yet still another embodiment, the present disclosure relates to a kit for use in an immunoassay. The kit for use in the immunoassay contains an antibody produced by murine hybridoma cell line 1-1175-509 and instructions for using said kit.

In yet still another embodiment, the present disclosure relates to a kit for use in an immunoassay. The kit for use in the immunoassay contains an antibody produced by murine hybridoma cell line 1-2169-715 and instructions for using said kit.

In yet still another embodiment, the present disclosure relates to a kit for use in an immunoassay. The kit for use in the immunoassay contains an antibody produced by murine hybridoma cell line 1-1175-509, an antibody produced by murine hybridoma cell line 1-2169-715 and instructions for using said kit.

In still yet another embodiment, the present disclosure relates to a method for determining concentration of myeloperoxidase (MPO) in a test sample. The method comprises the steps of:

(a) providing a test sample stored in a sample collection tube containing a MPO secretion inhibitor; and

(b) determining the concentration of MPO in the test sample.

In the above method, the MPO secretion inhibitor can be a salt of ethylene diamine tetraacetic acid. Additionally, the test sample used in the above method can be whole blood or a plasma sample.

Step (b) in the above method can be performed using a method selected from the group consisting of: a competitive immunoassay, a sandwich immunoassay, an enzyme-linked immunosorbent assay, an enzymatic assay and a clinical chemistry assay.

In the above method, the test sample can be stored at room temperature for a period of time up to about 8 hours. After storage at room temperature, the test sample can then be further stored at a temperature of from about 2° C. to about 8° C. for a period of up to about seven (7) days after processing.

Optionally, in the above method, the MPO secretion inhibitor can comprise a salt of citrate and the test sample can be stored at a temperature of from about 2° C. to about 8° C. for a period of up to about eight (8) hours prior to processing. Optionally, then said test sample can still further be stored at a temperature of from about 2° C. to about 8° C. for a period of up to about seven (7) days after processing.

In still yet another embodiment, the present disclosure relates to an improved method for determining the concentration of MPO in a human blood sample. Specifically, the improvement in this method comprises storing said sample in a sample collection tube containing a MPO secretion inhibitor at room temperature for a period of up to about 8 hours. In this method, the MPO secretion inhibitor can be a salt of ethylene diamine tetraacetic acid. Additionally, the human blood sample used in the above method can be whole blood or a plasma sample. Moreover, the concentration of MPO in the human blood sample can be determined by performing a method selected from the group consisting of: a competitive immunoassay, a sandwich immunoassay, an enzyme-linked immunosorbent assay, an enzymatic assay and a clinical chemistry assay. Additionally, after storage of the human blood sample at room temperature, the sample can then be further stored at a temperature of from about 2° C. to about 8° C. for a period up of from up to about seven (7) days after processing.

In still yet another embodiment, the present disclosure relates to an improved method for determining the concentration of MPO in a human blood sample. Specifically, the improvement in this method comprises storing said sample in a sample collection tube containing a salt of citrate at a temperature of from about 2° C. to about 8° C. for a period of up to eight (8) hours prior to processing. Optionally, then said test sample can still further be stored at a temperature of from about 2° C. to about 8° C. for a period of up to about seven (7) days after processing.

In still yet another embodiment, the present disclosure relates to a kit. The kit can comprise:

(a) a sample collection tube containing a MPO secretion inhibitor;

(b) at least one of the above described antibodies (e.g., an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438); and

(c) instructions for using said kit.

In still yet another embodiment, the present disclosure relates to an immunoassay for determining the concentration of MPO in a human peripheral blood sample. The immunoassay comprises the steps of:

(a) providing a human peripheral blood sample suspected of containing MPO stored in a sample collection tube containing a MPO secretion inhibitor;

(b) contacting a first capture antibody that binds to MPO with the human peripheral blood sample stored in a sample collection tube containing a MPO secretion inhibitor to form a first capture antibody-MPO complex, wherein the first capture antibody is an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438;

(c) contacting said human peripheral blood sample containing the first capture antibody-MPO complex with a second antibody that binds to MPO and that has been conjugated to a detectable label to form a second capture antibody-MPO-detection complex, wherein the second antibody is an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438, provided that the first capture antibody and the second antibody are not identical; and

(d) determining the amount of the capture antibody-MPO-detection complexes formed in step (b) by detecting the detectable label, wherein the amount of the second complexes formed is the amount of MPO contained in the human peripheral blood sample.

In the above immunoassay, the first capture antibody can be immobilized on a solid phase to produce an immobilized antibody. Also, in the above immunoassay, the first capture antibody can be an antibody produced by murine hybridoma cell line 1-1175-509 having A.T.C.C. Accession No. PTA-8437 and the second antibody can be an antibody produced by murine hybridoma cell line 1-2169-715 having A.T.C.C. Accession No. PTA-8438. In the above immunoassay, the MPO secretion inhibitor can be a salt of ethylene diamine tetraacetic acid.

Moreover, in the above immunoassay, the test sample can be whole blood or a plasma sample. In the above immunoassay, the test sample can be stored at room temperature for a period of time up to about 8 hours. After storage at room temperature, the test sample can be further stored at a temperature of from about 2° C. to about 8° C. for a period up to about seven (7) days after processing. Optionally, in the above method, the MPO secretion inhibitor can comprise a salt of citrate and the test sample can be stored at a temperature of from about 2° C. to about 8° C. for a period of up to about eight (8) hours prior to processing. Optionally, then said test sample can still further be stored at a temperature of from about 2° C. to about 8° C. for a period of up to about seven (7) days after processing.

In still yet another aspect, the present disclosure relates to a method of determining whether or not a subject is at risk of developing cardiovascular disease. Specifically, such a method can comprise the steps of:

(a) providing a test sample stored in a sample collection tube containing a MPO secretion inhibitor;

(b) determining the concentration of MPO in the test sample; and

(c) comparing the concentration of MPO in the test sample determined in step (b) with a predetermined level, wherein if the concentration of MPO determined in step (b) is lower than the predetermined level, then the subject is considered not to be at risk of developing cardiovascular disease and further wherein, if the concentration of MPO in the test sample determined in step (b) is higher then the predetermined level, then the subject is considered to be at risk of developing cardiovascular disease.

In the above method, the concentration of MPO in the test sample in step (b) can be determined using any of the previously described methods (e.g., methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies).

In still yet another aspect, the present disclosure relates to a method of diagnosing cardiovascular disease in a subject. The method can comprise the steps of:

(a) providing a test sample stored in a sample collection tube containing a MPO secretion inhibitor;

(b) determining the concentration of MPO in the test sample; and

(c) comparing the concentration of MPO in the test sample determined in step (b) with a predetermined level, wherein if the concentration of MPO determined in step (b) is lower than the predetermined level, then the subject would not be considered to have cardiovascular disease and further wherein, if the concentration of MPO in the test sample determined in step (b) is higher then a predetermined level, then the subject would be considered to have cardiovascular disease.

In the above method, the concentration of MPO in the test sample in step (b) can be determined using any of the previously described methods (e.g., methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies).

In still yet another aspect, the present disclosure relates to a method of monitoring the severity of cardiovascular disease in a subject. The method comprises the steps of:

(a) providing a test sample stored in a sample collection tube containing a MPO secretion inhibitor;

(b) determining the concentration of MPO in the test sample; and

(c) comparing the concentration of MPO in the test sample determined in step (b) with a predetermined level, wherein if the concentration of MPO determined in step (b) is lower than the predetermined level, the subject is determined to have a reduced severity of cardiovascular disease and further wherein if the concentration of MPO in the test sample determined in step (b) is higher than the predetermined level, the subject is determined to have an increased severity of cardiovascular disease.

In the above method, the cardiovascular disease is coronary artery disease, peripheral vascular disease, hypertension, myocardial infarction or heart failure. Moreover, in the above method, the concentration of MPO in the test sample in step (b) can be determined using any of the previously described methods (e.g., methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies).

In still yet another embodiment, the present disclosure relates to a method of monitoring the progression of cardiovascular disease in a subject. This method comprises the steps of:

(a) providing a test sample stored in a sample collection tube containing a MPO secretion inhibitor;

(b) determining the concentration of MPO in the test sample; and

(c) comparing the concentration of MPO in the test sample determined in step (b) with a predetermined level, wherein if the concentration of MPO determined in step (b) is lower than the predetermined level, the cardiovascular disease in the subject is determined not to have progressed or to that the subject has improved and if the concentration of MPO in the test sample determined in step (b) is higher than the predetermined level, the cardiovascular disease in the subject is determined to have progressed.

In the above method, the concentration of MPO in the test sample in step (b) can be determined using any of the previously described methods (e.g., methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies).

In still yet another embodiment, the present disclosure relates to a method of determining if a subject has suffered a cardiovascular complication as a result of administration to said subject of one or more pharmaceutical compositions. The method comprises the steps of:

(a) obtaining a first test sample from the subject before the subject has been administered one or more pharmaceutical compositions and storing said first test sample in a sample collection tube containing a MPO secretion inhibitor;

(b) determining the concentration of MPO in said sample;

(c) obtaining a second test sample from the subject after the subject has been administered one or more pharmaceutical compositions and storing said second test sample in a sample collection tube containing a MPO secretion inhibitor;

(d) determining the concentration of MPO in said second test sample; and

(e) comparing the concentration of MPO in step (b) with the concentration of MPO in step (d), wherein if the concentration of MPO determined in step (b) is unchanged when compared to the concentration of MPO determined in step (d), then the subject is determined not to have suffered a cardiovascular complication as a result of the administration of one or more pharmaceutical compositions and further wherein if the concentration of MPO determined in step (b) is changed when compared to the concentration of MPO in step (d), then the subject is determined to have suffered a cardiovascular complication as a result of the administration of one or more pharmaceutical compositions.

In the above method, the concentration of MPO in the test sample in step (b), step (d) or in both steps (b) and (d) can be determined using any of the previously described methods (e.g., methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies).

In still yet another embodiment, the present disclosure relates to a method of monitoring MPO levels in a subject receiving treatment with one or more pharmaceutical compositions. The method comprises the steps of:

(a) providing a first test sample from the subject before the subject has been administered one or more pharmaceutical compositions, wherein said test sample is stored in a sample collection tube containing a MPO secretion inhibitor;

(b) determining the concentration of MPO in the first test sample;

(c) comparing the concentration of MPO determined in step (b) with a predetermined level;

(d) treating the subject with one or more pharmaceutical compositions for a period of time if the comparison of the concentration of MPO determined in step (c) is that the concentration of MPO in the first test sample is greater than the predetermined level;

(e) providing a second and subsequent test samples from the subject after the subject has been administered one or more pharmaceutical compositions, wherein said test samples are stored in a sample collection tube containing a MPO secretion inhibitor;

(f) determining the concentration of MPO in the second and subsequent test samples;

(g) compare the concentrations of MPO determined in step (f) with the concentration of MPO determined in step (b), wherein if the concentrations of MPO determined in step (f) decrease when compared to the concentration of MPO determined in step (b), then the subject should continue to be administered the one or pharmaceutical compositions of step (d), further wherein, if the concentrations of MPO determined in step (f) are the same or increase when compared to the concentration of MPO determined in step (b), then the subject should be treated with a higher concentration of the one or more pharmaceutical compositions administered to the subject in step (d) or the subject should be treated with one or more pharmaceutical compositions that are different then the one or more pharmaceutical compositions administered to the subject in step (d).

In the above method, the concentration of MPO in the test sample in step (b), step (f) or in both steps (b) and (f) can be determined using any of the previously described methods (e.g., methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies).

### BRIEF DESCRIPTION OF THE FIGURES

FIG. 1 shows an antigen titration curve with the antibody concentration held at 0.3125 μg/mL generated as described in Example 9. In this FIG. 1, -- represents monoclonal antibody 1-1175-154, - |- represents monoclonal antibody 1-1519-450, - ♦- represents monoclonal antibody 1-2169-143, and - ▪- represents monoclonal antibody 1-2244-166.

FIG. 2 shows an antigen titration curve of the binding of various MPO binding pairs in an immunoassay as described in Example 9. In this FIG. 2, -x- represents the use of monoclonal antibody 1-1175-154 as the capture antibody and monoclonal antibody 1-1519-450 as the conjugate antibody, -- represents the use of monoclonal antibody 1-1519-450 as the capture antibody and monoclonal antibody 1-1175-154 as the conjugate antibody, -*- represents the use of monoclonal antibody 1-1175-154 as the capture antibody and monoclonal antibody 1-2244-166 as the conjugate antibody, and -- represents represents the use of monoclonal antibody 1-2169-143 as the capture antibody and monoclonal antibody 1-1175-154 as the conjugate antibody.

### DETAILED DESCRIPTION

The present disclosure relates to antibodies that can be used in myeloperoxidase (MPO) assays, such as, for example, immunoassays, to determine the amount or concentration of MPO in a test sample. The present disclosure also relates to an assay for determining the amount or concentration of MPO in a test sample using one or more of the antibodies of the present disclosure. In addition, the present disclosure also provides improved test sample handling methods that can be used to preserve original MPO levels in a test sample, thus allowing for a more accurate assessment of MPO levels and thus consequently, more accurate patient stratification and treatment selection. Finally, the present disclosure relates to an improved assay which employs a test sample wherein the original MPO levels in the a test sample have been preserved to allow for the accurate detection of MPO levels in said assay.

### A. Definitions

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

a) Antibody or Antibodies

As used herein, the terms “antibody” and “antibodies” refer to monoclonal antibodies, multispecific antibodies, human antibodies, humanized antibodies (fully or partially humanized), animal antibodies (such as, but not limited to, a bird (for example, a duck or goose), a shark or whale, a mammal, including a non-primate (for example, a cow, pig, camel, llama, horse, goat, rabbit, sheep, hamsters, guinea pig, cat, dog, rat, mouse, etc) or a non-human primate (for example, a monkey, such as a cynomologous monkey, a chimpanzee, etc), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies (including, for example, anti-Id antibodies to antibodies of the present disclosure), and functionally active epitope-binding fragments of any of the above. In particular, antibodies include immunoglobulin molecules and immunologically active fragments of immunoglobulin molecules, namely, molecules that contain an antigen binding site. Immunoglobulin molecules can be of any type (for example, IgG, IgE, IgM, IgD, IgA and IgY), class (for example, IgG₁, IgG₂, IgG₃, IgG₄, IgA₁, and IgA₂) or subclass. An antibody whose affinity (namely, K_{D}, k_{d} or kₐ) has been increased or improved via the screening of a combinatory antibody library that has been prepared using bio-display, is referred to herein as an “affinity maturated antibody”. For simplicity sake, an antibody against an analyte is frequently referred to herein as being either an “anti-analyte antibody”, or merely an “analyte antibody” (e.g., an MPO antibody or an anti-MPO antibody).

b) Binding Constants

The term “association rate constant”, “kₒₙ” or “kₐ” as used interchangeably herein, refers to the value indicating the binding rate of an antibody to its target antigen or the rate of complex formation between an antibody and antigen as shown by the equation below:

Antibody (“Ab”)+Antigen (“Ag”)→Ab−Ag.

The term “dissociation rate constant”, “k_{off}” or “k_{d}” as used interchangeably herein, refers to the value indicating the dissociation rate of an antibody from its target antigen or separation of Ab−Ag complex over time into free antibody and antigen as shown by the equation below:

Ab+Ag←Ab−Ag.

Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.) can also be used.

As used herein, the term “equilibrium dissociation constant” or “K_{D}” as used interchangeably, herein, refers to the value obtained by dividing the dissociation rate (k_{off}) by the association rate (kₒₙ). The association rate, the dissociation rate and the equilibrium dissociation constant are used to represent the binding affinity of an antibody to an antigen.

c) Blood or Blood Sample

As used herein, the terms “blood” or “blood sample”, are used interchangeably. The terms “blood” or “blood sample” refer to a whole blood sample serum or a plasma fraction derived therefrom. Preferably, the blood or blood sample is peripheral blood or a plasma fraction derived therefrom. Most preferably, the blood or blood sample is a human peripheral blood or plasma fraction derived therefrom.

d) Cardiovascular Disease

As used herein, the phrase “cardiovascular disease” refers to various clinical diseases, disorders or conditions involving the heart, blood vessels or circulation. The diseases, disorders or conditions may be due to atherosclerotic impairment of coronary, cerebral or peripheral arteries. Cardiovascular disease includes, but is not limited to, coronary artery disease, peripheral vascular disease, hypertension, myocardial infarction, heart failure, etc. With respect to heart failure, for example, “increased severity” of cardiovascular disease refers to the worsening of disease as indicated by increased NYHA classification, to, for example, Class III or Class IV and “reduced severity” of cardiovascular disease refers to an improvement of the disease as indicated by reduced NYHA classification, from, for example, class III or IV to class II or I.

e) Epitope or Epitopes

As used herein, the term “epitope” or “epitopes” refers to sites or fragments of a polypeptide or protein having antigenic or immunogenic activity in a subject. An epitope having immunogenic activity is a site or fragment of a polypeptide or protein that elicits an antibody response in an animal. An epitope having antigenic activity is a site or fragment of a polypeptide or protein to which an antibody immunospecifically binds as determined by any method well-known to those skilled in the art, for example by immunoassays.

f) Heart Failure

As used herein, the phrase “heart failure” refers to a condition in which the heart cannot pump blood efficiently to the rest of the body. Heart failure may be due to damage to the heart or narrowing of the arteries due to infarction, cardiomyopathy (primary or secondary), hypertension, coronary artery disease, valve disease, birth defects or infection. Heart failure can further be described as chronic, congestive, acute, decompensated, systolic or diastolic. The New York Heart Association (NYHA) classification describes the severity of the disease based on functional capacity of the patient; NYHA class can progress and/or regress based on treatment or lack of response to treatment.

g) Humanized Antibody

As used herein, the term “humanized” antibody refers to an immunoglobulin variant or fragment thereof, which is capable of binding to a predetermined antigen and which comprises framework regions having substantially the amino acid sequence of a human immunoglobulin and CDRs having substantially the amino acid sequence of a non-human immunoglobulin. Ordinarily, a humanized antibody has one or more amino acid residues introduced into it from a source that is non-human. In general, the humanized antibody will include substantially all of at least one, and typically two, variable domains (Fab, Fab′, F(ab′)₂, Fabc, Fv) in which all or substantially all of the CDR regions correspond to those of a non-human immunoglobulin and all or substantially all of the framework (FR) regions are those of a human immunoglobulin consensus sequence. The humanized antibody optimally comprises at least a portion of an immunoglobulin constant region (Fc), typically that of a human immunoglobulin. Generally, the antibody will contain both the light chain as well as at least the variable domain of a heavy chain. The humanized antibody can be selected from any class of immunoglobulins, including IgM, IgG, IgD, IgA and IgE, and any isotype, including IgG₁, IgG₂, IgG₃ and IgG₄. The humanized antibody may comprise sequences from more than one class or isotype, and selecting particular constant domains to optimize desired effector functions is within those skilled in the art

h) MPO Hybridoma

As used herein, the term “MPO hybridoma” (or merely “hybridoma”) refers to a particular hybridoma clone or subclone (as specified) that produces an anti-MPO antibody of interest. Generally, there may be some small variation in the affinity of antibodies produced by a hybridoma clone as compared to those from a subclone of the same type, e.g., reflecting purity of the clone. By comparison, it is well established that all hybridoma subclones originating from the same clone and further, that produce the anti-MPO antibody of interest, produce antibodies of identical sequence and/or identical structure.

i) Pharmaceutical Composition

As used herein, the term “pharmaceutical composition” refers to any agent or drug, whether a small molecule (e.g., a drug containing an active agent, typically a non-peptidic) or biologic (e.g., a peptide or protein based drug, including any with modifications, such as, but not limited to pegylation) that can be used to treat a subject suffering from a disease or condition that requires treatment. Examples of pharmaceutical compositions, include, but are not limited to, antineoplastics (chemotherapeutics), antidepressants (e.g., tricyclic antidepressants), multiple sclerosis drugs, anesthetics, interferons, hormones, HIV-antiviral drugs, hyperlipidemia drugs (including, but not limited to, niacin, fibrates (e.g., clofibrate, fenofibrate, fenofibric acid, simfrate, salts of fenofibric acid and any combinations thereof), ezetimibe, HMG-CoA reductase inhibitors (e.g., statins, such as, but not limited to rosuvastatin, simvastatin, and combinations thereof (including combinations with other hyperlipidemia drugs (e.g., simvastatin and ezetimibe)), anti-inflammatories, etc. as well as any combinations thereof.

j) Predetermined Level

As used herein, the term “predetermined level” refers generally at an assay cutoff value that is used to assess diagnostic results by comparing the assay results against the predetermined level, and where the predetermined level already that has been linked or associated with various clinical parameters (e.g., severity of disease, progression/nonprogression/improvement, etc.). The present disclosure provides exemplary predetermined levels, and describes the initial linkage or association of such levels with clinical parameters for exemplary immunoassays as described herein. However, it is well known that cutoff values may vary dependent on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this description. Whereas the precise value of the predetermined level (cutoff) may vary between assays, the correlations as described herein should be generally applicable.

k) Sample Collection Tube

As used herein, the terms “sample collection tube” or “sample tube”, are used interchangeable. As used herein, the terms “sample collection tube” or “sample tube” refer to any type of container used to collect and store a test sample, such as a blood sample, for shipment to an analytical processing site. Sample collection tubes can be made with any suitable material known in the art (e.g., plastic or glass). For example, the sample collection tube can be made of a suitable plastic material that is non-reactive with the stabilizing agents and does not interfere with the test sample. Preferred plastic material include any type of polyethylene terepththlate (PET) or polypropylene. An example of plastic sample collection tube are the sample collection tubes known as VACUETTE®, available from Greiner Bio-One GmbH (Kremsmunster, Austria) and BD VACUTAINER® available from BD (Becton, Dickinson and Company, Franklin Lakes, N.J.). Particularly preferred are the plastic EDTA-containing VACUTAINER® tubes (BD, Franklin Lakes, N.J.), especially lavendar-top tubes tube spray-coated with EDTA (namely, VACUTAINER® K₂EDTA or K₃EDTA tubes) and plasma preparation tubes (PPT) with EDTA. Sample collection tubes can also contain other chelators that function similar to EDTA, such as, but not limited to, ethylene glycol tetraacetic acid (EGTA) or glycol ether diamine tetraacetic acid, nitrilotriacetic acid (NTA) or diethylenetriaminepentaacetic acid (DTPA). Alternatively, the sample collection tube can be made of glass or siliconized glass.

Sample collection tubes can be made by any process known in the art, such as, for example, injection molding. Moreover, the sample tube can be of any design, including nested designs, such as described in U.S. Pat. No. 6,910,597, M. Iskra, “Collection Container Assembly”. Additionally, the sample collection tubes may be coated with a clot activator (such as, for example, silicon and/or micronized silica particles). In addition to the clot activator, these tubes may also contain a gel. Alternatively, the sample collection tube may contain a gel and other materials (such as lithium heparin) to facilitate the processing of the test sample. Examples of such sample collection tubes are the BD VACUTAINER® Serum Tubes, the BD VACUTAINER® SST™ tubes (which are plastic tubes that contain a gel and a clot activator) and BD VACUTAINER® PST™ tubes (which are plastic tubes that contain a gel and lithium heparin).

l) Subject or Patient

As used herein, the terms “subject” and “patient” are used interchangeably. As used herein, the terms “subject” and “subjects” refer to an animal, in one aspect, a bird (for example, a duck or goose), in another aspect, a shark or whale, or in a further aspect, a mammal including, a non-primate (for example, a cow, pig, camel, llama, horse, goat, rabbit, sheep, hamsters, guinea pig, cat, dog, rat, and mouse) and a primate (for example, a monkey, such as a cynomolgous monkey, chimpanzee, and a human).

m) Test Sample

As used herein, the term “test sample” generally refers to a biological material being tested for and/or suspected of containing an analyte of interest. The test sample may be derived from any biological source, such as, a physiological fluid, including, but not limited to, whole blood (hemolyzed or unhemolyzed), serum, plasma, red blood cells (erythrocytes), white blood cells (leukocytes including granulocytes such as neutrophils, eosinophils and basophils, and including lymphoid cells such as lymphocytes and monocytes), and other blood cells or forms of blood (e.g., platelets, lymph), interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. The test sample may be tested immediately following its collection (i.e., fresh) or following some period of storage under appropriate storage conditions. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte.

The terminology used herein is for the purpose of describing particular embodiments only and is not otherwise intended to be limiting.

### B. MPO Antibodies

The present disclosure provides antibodies that specifically bind to MPO. More specifically, the inventors have discovered antibodies that bind to distinct epitope groupings on MPO. In fact, the inventors of the present disclosure have discovered that human MPO contains at least eight (8) distinct epitope groups.

In particular, in one aspect, the present disclosure provides for isolated antibodies that bind to one distinct epitope group on MPO, referred to herein as epitope group number 3.

In another aspect, the present disclosure relates to murine hybridoma cell line 1-1175-509 (also referred to as “MPO 1-1175-509”) having A.T.C.C. Accession No. PTA-8437, deposited on May 16, 2007.

In yet another aspect, the present disclosure relates to an antibody made from DNA extracted from murine hybridoma cell line 1-1175-509 having A.T.C.C. Accession No. PTA-8437.

In still yet another aspect, the present disclosure relates to an antibody produced by murine hybridoma cell line 1-1175-509 having A.T.C.C. Accession No. PTA-8437, deposited on May. The antibody produced by murine hybridoma cell line 1-1175-509 can bind to epitope group number 3 on MPO.

In particular, in one aspect, the present disclosure provides for isolated antibodies that bind to one distinct epitope group on MPO, referred to herein as epitope group number 5.

In yet another aspect, the present disclosure relates to murine hybridoma cell line 1-2169-715 (also referred to as “MPO 1-2169-715”) having A.T.C.C. Accession No. PTA-8438, deposited on May 16, 2007.

In still another aspect, the present disclosure relates to antibody made from DNA extracted from murine hybridoma cell line 1-2169-715 having A.T.C.C. Accession No. PTA-8438.

In still yet another aspect, the present disclosure relates to an antibody produced by murine hybridoma cell line 1-2169-715 having A.T.C.C. Accession No. PTA-8438, deposited on May. The antibody produced by murine hybridoma cell line 1-2169-715 can bind to epitope group number 5 on MPO.

### C. Methods of Making and Using MPO Antibodies

The antibodies of the present disclosure can be made using a variety of different techniques known in the art. For example, polyclonal and monoclonal antibodies against MPO can be raised by immunizing a suitable subject (such as, but not limited to, a rabbit, goat, mouse or other mammal) with an immunogenic preparation which contains a suitable immunogen, such as purified MPO antigen. For example, a suitable immunogen can be MPO purified from human neutrophils, which is commercially available from Athens Research & Technology (Athens, Ga.).

The antibodies raised in the subject can then be screened to determine if the antibodies bind to MPO. Such antibodies can be further screened using the methods described herein (See, Example 9). For example, these antibodies can be assayed to determine if they bind to epitope group number 3 or epitope group number 5. Suitable methods to identify an antibody with the desired characteristics are described herein (See, Example 10).

The unit dose of immunogen (namely, the purified protein, or recombinantly produced human MPO protein) and the immunization regimen will depend upon the subject to be immunized, its immune status, and the body weight of the subject. To enhance an immune response in the subject, an immunogen can be administered with an adjuvant, such as Freund's complete or incomplete adjuvant, Ribi's adjuvant or any combinations thereof.

Immunization of a subject with an immunogen as described above induces a polyclonal antibody response. The antibody titer in the immunized subject can be monitored over time by standard techniques such as an ELISA using an immobilized antigen, namely, MPO.

Human monoclonal antibodies can be produced by introducing an antigen into immune deficient mice that have been engrafted with human antibody-producing cells or tissues (for example, human bone marrow cells, peripheral blood lymphocytes (PBL), human fetal lymph node tissue, or hematopoietic stem cells). Such methods include raising antibodies in SCID-hu mice (See, for example, WO 93/05796, U.S. Pat. No. 5,411,749; or McCune et al., Science, 241:1632-1639 (1988)) or Rag-1/Rag-2 deficient mice. Human antibody-immune deficient mice are also commercially available. For example, Rag-2 deficient mice are available from Taconic Farms (Germantown, N.Y.).

Monoclonal antibodies can be generated by immunizing a subject with an immunogen. At the appropriate time after immunization, for example, when the antibody titers are at a sufficiently high level, antibody producing cells can be harvested from an immunized animal and used to prepare monoclonal antibodies using standard techniques. For example, the antibody producing cells can be fused by standard somatic cell fusion procedures with immortalizing cells such as myeloma cells to yield hybridoma cells. Such techniques are well known in the art, and include, for example, the hybridoma technique as originally developed by Kohler and Milstein, Nature, 256:495-497 (1975)), the human B cell hybridoma technique (Kozbar et al., Immunology Today, 4:72 (1983)), and the EBV-hybridoma technique to produce human monoclonal antibodies (Cole et al., Monoclonal Antibodies and Cancer Therapy, Alan R. Liss, Inc. pp. 77-96 (1985)). The technology for producing monoclonal antibody hybridomas is well known to those skilled in the art.

Monoclonal antibodies can also be made by harvesting antibody producing cells, for example, splenocytes, from transgenic mice expressing human immunoglobulin genes and which have been immunized with MPO. The splenocytes can be immortalized through fusion with human myelomas or through transformation with Epstein-Barr virus (EBV). These hybridomas can be made using human B cell-or EBV-hybridoma techniques described in the art (See, for example, Boyle et al., European Patent Publication No. 0 614 984).

Hybridoma cells producing a monoclonal antibody which specifically binds to MPO are detected by screening the hybridoma culture supernatants by, for example, screening to select antibodies that specifically bind to the immobilized MPO, or by testing the antibodies as described herein to determine if the antibodies have the desired characteristics, namely, the ability to bind to MPO at the unique epitope groups (namely, MPO epitope group 3 or 5) described herein. After hybridoma cells are identified that produce antibodies of the desired specificity, the clones may be subcloned, e.g., by limiting dilution procedures, for example the procedure described by Wands et al. (Gastroenterology 80:225-232 (1981)), and grown by standard methods.

Hybridoma cells that produce monoclonal antibodies that test positive in the screening assays described herein can be cultured in a nutrient medium under conditions and for a time sufficient to allow the hybridoma cells to secrete the monoclonal antibodies into the culture medium, to thereby produce whole antibodies. Tissue culture techniques and culture media suitable for hybridoma cells are generally described in the art (See, for example, R. H. Kenneth, in Monoclonal Antibodies: A New Dimension In Biological Analyses, Plenum Publishing Corp., New York, N.Y. (1980)). Conditioned hybridoma culture supernatant containing the antibody can then be collected. The monoclonal antibodies secreted by the subclones optionally can be isolated from the culture medium by conventional immunoglobulin purification procedures such as, for example, protein A chromatography, hydroxylapatite chromatography, gel electrophoresis, dialysis, or affinity chromatography.

Monoclonal antibodies can be engineered by constructing a recombinant combinatorial immunoglobulin library and screening the library with the MPO. Kits for generating and screening phage display libraries are commercially available (See, for example, the Pharmacia Recombinant Phage Antibody System, Catalog No. 27-9400-01; and the Stratagene SurfZAP Phage Display Kit, Catalog No. 240612). Likewise, yeast display vectors are known in the art and are commercially available (for example, pYD1 available from Invitrogen Corp., Carlsbad, Calif.). Briefly, the antibody library is screened to identify and isolate phages or yeast cells that express an antibody that specifically binds to MPO. Preferably, the primary screening of the library involves screening with an immobilized MPO.

Following screening, the display phage or yeast is isolated and the polynucleotide encoding the selected antibody can be recovered from the display phage or yeast (for example, from the phage or yeast genome) and subcloned into other expression vectors (e.g., into Saccharomyces cerevesiae cells, for example EBY100 cells (Invitrogen Corporation, Carlsbad, Calif.)) by well known recombinant DNA techniques. The polynucleotide can be further manipulated (for example, linked to nucleic acid encoding additional immunoglobulin domains, such as additional constant regions) and/or expressed in a host cell.

Alternatively, recombinant forms of antibodies, such as chimeric and humanized antibodies, can also be prepared to minimize the response by a human patient to the antibody. When antibodies produced in non-human subjects or derived from expression of non-human antibody genes are used therapeutically in humans, they are recognized to varying degrees as foreign, and an immune response may be generated in the patient. One approach to minimize or eliminate this immune reaction is to produce chimeric antibody derivatives, namely, antibody molecules that combine a non-human animal variable region and a human constant region. Such antibodies retain the epitope binding specificity of the original monoclonal antibody, but may be less immunogenic when administered to humans, and therefore more likely to be tolerated by the patient.

Chimeric monoclonal antibodies can be produced by recombinant DNA techniques known in the art. For example, a gene encoding the constant region of a non-human antibody molecule is substituted with a gene encoding a human constant region (See, for example, PCT Patent Publication PCT/US86/02269, European Patent Application 184,187 or European Patent Application 171,496).

A chimeric antibody can be further “humanized” by replacing portions of the variable region not involved in antigen binding with equivalent portions from human variable regions. General reviews of “humanized” chimeric antibodies can be found in Morrison, S. L., Science, 229:1202-1207 (1985) and in Oi et al., BioTechniques, 4-214 (1986). Such methods include isolating, manipulating, and expressing the nucleic acid sequences that encode all or part of an immunoglobulin variable region from at least one of a heavy or light chain. The cDNA encoding the humanized chimeric antibody, or fragment thereof, can then be cloned into an appropriate expression vector. Suitable “humanized” antibodies can be alternatively produced by complementarity determining region (CDR) substitution (See, for example, U.S. Pat. No. 5,225,539; Jones et al., Nature, 321:552-525 (1986); Verhoeyan et al., Science 239:1.534 (1988); and Beidler et al., J. Immunol., 141:4053-4060 (1988)).

Epitope imprinting can also be used to produce a “human” antibody polypeptide dimer that retains the binding specificity of the antibodies (for example, hamster antibodies) specific for MPO. Briefly, a gene encoding a non-human variable region (VH) with specific binding to an antigen and a human constant region (CH1), is expressed in E. coli and infected with a phage library of human Vλ.Cλ genes. Phage displaying antibody fragments are then screened for binding to MPO. Selected human Vλ genes are recloned for expression of Vλ.Cλ. chains and E. coli harboring these chains are infected with a phage library of human VHCH1 genes and the library is subject to rounds of screening with antigen coated tubes (See, WO 93/06213).

In another aspect, the present disclosure contemplates that the antibody is an antibody fragment. For example, the antibody fragment can include, but is not limited to, a Fab, a Fab′, a Fab′-SH fragment, a di-sulfide linked Fv, a single chain Fv (scFv) and a F(ab′)₂ fragment. Various techniques are known to those skilled in the art for the production of antibody fragments. For example, such fragments can be derived via proteolytic digestion of intact antibodies (See, for example, Morimoto et al., J. Biochem. Biophys. Methods, 24:107-117 (1992) and Brennan et al., Science, 229:81 (1985)) or produced directly by recombinant host cells. For example, Fab′-SH fragments can be directly recovered from E. coli and chemically coupled to form F(ab′)₂ fragments (See, Carter et al., Bio/Technology, 10:163-167 (1992)). In another embodiment, the F(ab′)₂ is formed using the leucine zipper GCN4 to promote assembly of the F(ab′)₂ molecule. Alternatively, Fv, Fab or F(ab′)₂ fragments can be isolated directly from recombinant host cell culture. Single chain variable region fragments (scFv) are made by linking light and/or heavy chain variable regions by using a short linking peptide (See, Bird et al. Science, 242:423-426 (1998)). An example of a linking peptide is GPAKELTPLKEAKVS (SEQ ID NO: 1). Linkers can in turn be modified for additional functions, such as attachment of drugs or attachment to solid supports. Examples of other linker sequences that can be used in the present disclosure can be found in Bird et al., Science, 242:423-426 (1988), Huston et al., Proc. Natl. Acad. Sci. USA, 85:5879-5883 (1988) and McCafferty et al., Nature, 348:552-554 (1990).

The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art. Moreover, other forms of single chain antibodies, such as diabodies are also contemplated by the present disclosure. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (See, for example, Holliger, P., et al., Proc. Natl. Acad. Sci. USA, 90:6444-6448 (1993); Poljak, R. J., et al., Structure, 2:1121-1123 (1994)).

The antibodies of the present disclosure have a variety of uses. More specifically, the antibodies of the present disclosure can be used as one or more capture antibodies, one or more conjugate antibodies or as both one or more capture antibodies and one or more conjugate antibodies in immunoassays to detect the presence of MPO in a test sample.

### D. MPO Test Sample Collection and Improved Test Sample Handling Methods

The present disclosure also provides improved test sample handling methods that can be used to preserve original MPO levels obtained from a collected test sample. Specifically, these improved test sampling methods involve storing a test sample obtained from a subject in a sample collection tube containing a leukocyte MPO secretion inhibitor. Preferably, the methods involve storing a blood sample (e.g., a peripheral blood sample) obtained from a subject in a sample tube containing a leukocyte MPO secretion inhibitor. These methods have significant capability to provide improved determination of blood concentration levels of MPO and thus enable more accurate preservation of and measurement of MPO levels and consequently more accurate patient stratification and treatment selection. Additionally, test samples that have had their original MPO levels preserved according to the methods described herein can be used in assays to determine the concentration of MPO in a test sample. As will be discussed in more detail herein, determining the concentration of MPO in a test sample involves: (a) providing a test sample obtained from a subject, preferably, a peripheral blood sample, stored in a sample collection tube containing a MPO secretion inhibitor; and (b) determining the concentration of MPO in the test sample. Methods for collecting, handling and processing test samples (such as whole blood, serum and plasma, and other body fluids) that can be used in the practice of the methods of the present disclosure are well known in the art.

As mentioned above, preservation of original test sample (e.g., blood or plasma) MPO levels from collection time until measurement can be obtained by the use of the leukocyte MPO secretion inhibitor. A leukocyte MPO secretion inhibitor can be any reagent added to the test sample on or shortly after collection into the sample collection tube (e.g., a plasma collection tube), that inhibits release of MPO from leukocytes present in the test sample (e.g., serum or plasma) particularly, release at room temperature. The inhibition of MPO release by the leukocytes (by the inhibitor) occurs prior to the processing (e.g., by centrifugation methods), of the test sample into various fractions (e.g., a plasma fraction, a buffy coat interface (or fraction) next to the plasma fraction (where the leukocytes are) and a red blood cell fraction). Examples of leukocyte MPO secretion inhibitors that can be used include, but are not limited to, salts of EDTA (including, but not limited to sodium or potassium salts) or salts of citrate (including, but not limited to sodium citrate). Preferred EDTA salts include, but are not limited to, dipotassium and tripotassium salts. Preferred citrate salts are sodium citrate. The amount of leukocyte MPO secretion inhibitor to be added can be determined by one skilled in the art. Specifically, the amounts used in commercially available sample collection tubes for whole blood are acceptable. Examples of such sample collection tubes that can be used include, but are not limited to, EDTA-containing tubes, e.g., plastic EDTA-containing VACUTAINER® tubes (such as sold by BD, Franklin Lakes, N.J.), especially lavendar-top tubes tube spray-coated with EDTA (namely, VACUTAINER® K₂EDTA or K₃EDTA tubes) and plasma preparation tubes (PPT) with EDTA.

After collection of the test sample into a sample collection tube (referred to herein as a “collected test sample”) with a leukocyte MPO secretion inhibitor, the sample collection tube may be stored for a period of time not longer than about 8 hours at room temperature. The inventors of the present disclosure have found that the levels of MPO in a collected test sample can be preserved (e.g., maintained) at room temperature for up to about 8 hours in sample collection tubes containing a MPO secretion inhibitor prior to processing. Particularly good results were obtained using sample collection tubes that contained salts of EDTA. Specifically, the inventors found that the use of salts of EDTA preserves the integrity of the leukocytes in the test sample thereby preventing MPO leakage out of these cells and thus preserving the levels of MPO in the test sample. Alternatively, test samples in sample collection tubes containing salts of citrate can be stored at other than room temperature, namely at a temperature of from about 2° C. to about 8° C., for a period of from up to about eight (8) hours prior to processing.

After the a period of storage at room temperature or at a temperature of from about 2° C. to about 8° C., the collected test sample can be centrifuged and then placed on ice or stored at a temperature in a range of about 2° C. to about 8° C., until the collected test sample is processed for MPO determination. Although it is possible to use a temperature range lower than about 2° C. to about 8° C., this is not preferred and should be avoided to lessen the likelihood the sample will freeze. Additionally, sample collection tube can be further stored at a temperature in a range of about 2° C. to about 8° C. for a period of up to about seven (7) days after processing (e.g., such as after centrifugation and/or plasma separation). This is especially preferred when the sample collection tubes contain salts of EDTA or salts of citrate.

Prior to processing, the preservation status of the test sample can be checked, reviewed or verified. The preservation status (namely, the MPO preservation conditions) of the test sample can be checked, reviewed or verified in a number of different ways. For example, the temperature and time at which the test sample has been stored can be assessed to determine whether or not the preservation conditions are unacceptable for further processing and use of the test sample in an assay. For example, unacceptable preservation conditions would be storage of the test sample in a sample collection tube that does not contain a MPO secretion inhibitor or storage of the test sample in a sample collection tube containing a leukocyte MPO secretion inhibitor at room temperature for a period of over about 8 hours prior to processing. Alternatively, the test sample can be analyzed for the presence of lysis of leukocytes in the test sample. The presence of such lysis would indicate that the preservation conditions are unacceptable and that the test sample is unacceptable for use further in an assay. Checking the MPO preservation status of a test sample can be automated in an analytical instrument, such as an automated immunoassay analyzer. As will be discussed in more detail herein, the automated immunoassay analyzer can be programmed to check on the MPO preservation conditions used for the test sample, and where potentially unacceptable preservation conditions are determined as present, the analyzer can be programmed to send an error message that it is unable to perform the MPO assay, because of the likelihood of inaccurately high results.

Any processing steps known in the art can be used to produce a plasma fraction from a test sample. For example, centrifugation can be used.

### E. MPO Assays

The present disclosure also relates to assays for determining MPO concentration in a test sample obtained from a subject. Assays contemplated include immunoassays (such as sandwich and competitive immunoassays), clinical chemistry assays and enzymatic assays. Preferably the MPO measurement is done using an immunoassay, and more preferably, a sandwich immunoassay, which will be discussed in more detail herein.

Assays for determining MPO concentration in a test sample obtained from a subject can comprise the steps of: (a) providing a test sample obtained from a subject; and (b) determining the concentration of MPO in the test sample. Preferably, the test sample obtained from a subject is a peripheral blood sample. In the assays of the present disclosure, the peripheral blood sample has been stored in a sample collection tube containing a MPO secretion inhibitor. Optionally, when the test sample obtained from a subject has been stored in a sample collection tube containing a MPO secretion inhibitor, the assay can also comprise an intermediate or additional step. This intermediate or additional step involves checking the preservation conditions in the test sample. This intermediate or additional step of checking, reviewing or verifying the preservation conditions in the test sample must be performed prior to determining the concentration of MPO in the test sample. As discussed previously herein, this intermediate or additional step is particularly advantageous for use with an automated analytical instrument, such as an automated immunoassay analyzer. The analyzer can be programmed to check, review or verify the preservation conditions in the test sample as well as identify unacceptable MPO storage conditions, such as storage of the collected test sample at room temperature for longer than about 8 hours or further storage of the collected test sample at a temperature of or 2° C. to 8° C. for over about 7 days before test sample centrifugation and plasma separation. If unacceptable MPO levels or MPO storage conditions are determined as present, the analyzer can be programmed to send an error message that it is unable to perform the MPO assay, because of the likelihood of inaccurately high results. In this aspect, the analyzer would proceed to process a test sample stored in a collection tube containing a MPO secretion inhibitor and stored at room temperature for no longer than about 8 hours.

A specific type of assay that can be performed for determining MPO concentration is an immunoassay. Immunoassays can be conducted using any format known in the art, such as, but not limited to, a sandwich format, a competitive inhibition format (including both forward or reverse competitive inhibition assays) or in a fluorescence polarization format. As mentioned above, preferably, the immunoassay is in a sandwich format. Specifically, in one aspect of the present disclosure, at least two antibodies are employed to separate and quantify human MPO in a test sample. More specifically, the at least two antibodies bind to certain epitopes of MPO forming an immune complex which is referred to as a “sandwich”. Generally, in the immunoassays one or more antibodies can be used to capture the MPO in the test sample (these antibodies are frequently referred to as a “capture” antibody or “capture” antibodies) and one or more antibodies can be used to bind a detectable (namely, quantifiable) label to the sandwich (these antibodies are frequently referred to as the “detection antibody”, “detection antibodies”, a “conjugate” or “conjugates”). In a sandwich assay, it is preferred that both antibodies binding to the MPO are not diminished by the binding of any other antibody in the assay to its respective binding site. In other words, antibodies should be selected so that the one or more first antibodies brought into contact with a test sample or test sample extract suspected of containing MPO do not bind to all or part of the binding site recognized by the second or subsequent antibodies, thereby interfering with the ability of the one or more second detection antibodies to bind to MPO.

Excellent immunoassays, particularly, sandwich assays, can be performed using the antibodies of the present disclosure as the capture antibodies, detection antibodies or as capture and detection antibodies. For example, at least one of the antibodies of the present disclosure (such as antibody produced by murine hybridoma cell line 1-1175-509 or an antibody produced by murine hybridoma cell line 1-2169-715 or a combination of an antibody produced by murine hybridoma cell line 1-1175-509 and an antibody produced by murine hybridoma cell line 1-2169-715) can be used as a first capture antibody and other commercially available antibodies can be used as the detection antibodies. Alternatively, in more than one capture antibody is being used, then the antibodies of the present disclosure can be used as a second or subsequent capture antibody. Alternatively, if one of the antibodies of the present disclosure is being used as a capture antibody, a different antibody (other than an antibody of the present disclosure, namely, other commercially available antibodies) can be used as a second capture antibody. Alternatively, the antibodies of the present disclosure can be used only as detection antibodies and not as capture antibodies with other commercially available antibodies being used as the capture antibodies. Still in another alternative, the antibodies of the present disclosure can be used as both capture and detection antibodies. For example, an antibody (or an antibody fragment thereof) produced by murine hybridoma cell line 1-1175-509 can be used as a capture antibody and an antibody (or a antibody fragment thereof) produced by murine hybridoma cell line 1-2169-715 can be used as a detection antibody. Alternatively, an antibody (or an antibody fragment thereof) produced by murine hybridoma cell line 1-2169-715 can be used as a capture antibody and an antibody (or an antibody fragment thereof) produced by hybridoma cell line 1-1175-509 can be used as a detection antibody.

The test sample being tested for (for example, suspected of containing) MPO can be contacted with at least one capture antibody (or antibodies) and at least one detection antibody (which is either a second detection antibody or a third detection antibody) either simultaneously or sequentially and in any order. For example, the test sample can be first contacted with at least one capture antibody and then (sequentially) with at least one detection antibody. Alternatively, the test sample can be first contacted with at least one detection antibody and then (sequentially) with at least one capture antibody. In yet another alternative, the test sample can be contacted simultaneously with a capture antibody and a detection antibody. The test sample being tested can be stored is a sample collection tube containing a leukocyte MPO secretion inhibitor as described previously herein. Alternatively, the test sample does not need to have been stored in a sample collection tube containing a leukocyte MPO secretion inhibitor.

In the sandwich assay format, a test sample suspected of containing MPO is first brought into contact with an at least one first capture antibody under conditions which allow the formation of a first antibody-MPO complex. If more than one capture antibody is used, a first multiple capture antibody-MPOcomplex is formed. In a sandwich assay, the antibodies, preferably, the at least one capture antibody, are used in molar excess amounts of the maximum amount of MPO expected in the test sample. For example, from about 5 μg/mL to about 1 mg/mL of antibody per mL of buffer (e.g., microparticle coating buffer) can be used.

Optionally, prior to contacting the test sample with the at least one capture antibody (for example, the first capture antibody), the at least one capture antibody can be bound to a solid support or solid phase which facilitates the separation the first antibody-MPO complex from the test sample. Any solid support known in the art can be used, including but not limited to, solid supports made out of polymeric materials in the forms of wells of a reaction tray, test tubes or beads (for example, polystyrene beads, magnetic beads), nitrocellulose strips, membranes, microparticles (for example, latex particles, sheep and DURACYTES® (Abbott Laboratories, Abbott Park, Ill.; DURACYTES® are red blood cells that have been “fixed” by pyruvic aldehyde and formaldehyde)).

The solid phase also can comprise any suitable porous material with sufficient porosity to allow access by detection antibodies and a suitable surface affinity to bind antigens. Microporous structures generally are preferred, but materials with gel structure in the hydrated state may be used as well. Such useful solid supports include, but are not limited to, nitrocellulose and nylon. Such porous solid supports are preferably in the form of sheets of thickness from about 0.01 to 0.5 mm, preferably about 0.1 mm. The pore size may vary within wide limits, and preferably is from about 0.025 to about 15 microns, especially from about 0.15 to about 15 microns. The surface of such supports may be activated by chemical processes which cause covalent linkage of the antigen or antibody to the support. The irreversible binding of the antigen or antibody is obtained, however, in general, by adsorption on the porous material by poorly understood hydrophobic forces.

The antibody (or antibodies) can be bound to the solid support or solid phase by adsorption, by covalent bonding using a chemical coupling agent or by other means known in the art, provided that such binding does not interfere with the ability of the antibody to bind MPO. Alternatively, the antibody (or antibodies) can be bound with microparticles that have previously coated with streptavidin or biotin (for example, using Power-Bind™-SA-MP streptavidin coated microparticles, available from Seradyn, Indianapolis, Ind.). Alternatively, the antibody (or antibodies) can be bound using microparticles that have been previously coated with anti-species specific monoclonal antibodies. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample being tested for and/or suspected of containing MPO is brought into contact with the at least one capture antibody (for example, the first capture antibody), the mixture is incubated in order to allow for the formation of a first antibody (or multiple antibody)-MPO complex. The incubation can be carried out at a pH of from about 4.5 to about 10.0, at a temperature of from about 2° C. to about 45° C., and for a period from at least about one (1) minute to about eighteen (18) hours, preferably from about 1 to 20 minutes, most preferably from about 2-6 minutes. The immunoassay described herein can be conducted in one step (meaning the test sample, at least one capture antibody and at least one detection antibody are all added sequentially or simultaneously to a reaction vessel) or in more than one step, such as two steps, three steps, etc.

After formation of the (first or multiple) capture antibody-MPO complex, the complex is then contacted with at least one detection antibody (under conditions which allow for the formation of a (first or multiple) capture antibody-MPO-second antibody detection complex). The at least one detection antibody can be the second, third, fourth, etc. antibodies used in the immunoassay. If the capture antibody-MPO complex is contacted with more than one detection antibody, then a (first or multiple) capture antibody-MPO-(multiple) detection antibody complex is formed. As with the capture antibody (e.g., the first capture antibody), when the at least second (and subsequent) detection antibody is brought into contact with the capture antibody-MPO complex, a period of incubation under conditions similar to those described above is required for the formation of the (first or multiple) capture antibody-MPO-(second or multiple) detection antibody complex. Preferably, at least one detection antibody contains a detectable label. The detectable label can be bound to the at least one detection antibody (e.g., the second detection antibody) prior to, simultaneously with or after the formation of the (first or multiple) capture antibody-MPO-(second or multiple) detection antibody complex. Any detectable label known in the art can be used. For example, the detectable label can be a radioactive label, such as, ³H, ¹²⁵I, ³⁵S, ¹¹C, ³²P, ³³P, an enzymatic label, such as horseradish peroxidase, alkaline phosphatase, glucose 6-phosphate dehydrogenase, etc., a chemiluminescent label, such as, acridinium esters, luminol, isoluminol, thioesters, sulfonamides, phenanthradinium esters, etc. a fluorescence label, such as, fluorescein (5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2 ed., Springer Verlag, N.Y. (1997) and in Haugland, Handbook of Fluorescent Probes and Research Chemicals (1996), which is a combined handbook and catalogue published by Molecular Probes, Inc., Eugene, Oreg.

The detectable label can be bound to the antibodies either directly or through a coupling agent. An example of a coupling agent that can be used is EDAC (1-ethyl-3-(3-dimethylaminopropyl) carbodiimide, hydrochloride) that is commercially available from Sigma-Aldrich, St. Louis, Mo. Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as, N10-(3-sulfopropyl)-N-(3-carboxypropyl)-acridinium-9-carboxamide, otherwise known as CPSP-Acridinium Ester or N10-(3-sulfopropyl)-N-(3-sulfopropyl)-acridinium-9-carboxamide, otherwise known as SPSP-Acridinium Ester.

The (first or multiple) capture antibody-MPO-(second or multiple) detection antibody complex can be, but does not have to be, separated from the remainder of the test sample prior to quantification of the label. For example, if the at least one capture antibody (e.g., the first capture antibody) is bound to a solid support or solid phase, such as, but not limited to a well of a reaction tray, a bead or a microparticle, separation can be accomplished by removing the fluid (of the test sample) from contact with the solid support. Alternatively, if the at least first capture antibody is bound to a solid support it can be simultaneously contacted with the MPO-containing sample and the at least one second detection antibody to form a first (multiple) antibody-MPO-second (multiple) antibody complex, followed by removal of the fluid (test sample) from contact with the solid support. If the at least one first capture antibody is not bound to a solid support, then the (first or multiple) capture antibody-MPO-(second or multiple) detection antibody complex does not have to be removed from the test sample for quantification of the amount of the label.

After formation of the labeled capture antibody-MPO-detection antibody complex (e.g., the first capture antibody-MPO-second detection antibody complex), the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. Once the amount of the label in the complex has been quantified, the concentration of MPO in the test sample is determined by use of a standard curve that has been generated using serial dilutions of MPO of known concentration. Other than using serial dilutions of MPO, the standard curve can be generated gravimetrically, by mass spectroscopy and by other techniques known in the art.

Any suitable control composition can be used in the MPO immunoassays. The control compositions generally comprise the MPO antigen to be assayed for along with any desirable additives. A preferred control MPO antigen is available commercially from Athens Research and Technology Inc. (Athens, Ga.).

The MPO assays described herein can be used for diagnosing cardiovascular disease in a subject. Specifically, such assays involve providing a test sample obtained from a subject (which may or may not have been stored in a sample collection tube containing a MPO secretion inhibitor as described previously herein in Section D). The concentration of MPO in the test sample can then determined using any of the MPO assays described herein (e.g., using the methods described in this Section E, namely, methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies). Once the concentration of MPO in the test sample is determined it can be compared to a predetermined level to determine whether or not the subject is suffering from cardiovascular disease. Specifically, if the concentration of MPO in the test sample is lower or the same as a predetermined level, then the subject would be considered not to have cardiovascular disease. However, if the concentration of MPO in the test sample were higher then a predetermined level, then the subject would be considered to have cardiovascular disease.

The concentration of MPO in a test sample determined using the method described herein can also be used to determine whether or not a subject is at risk of developing cardiovascular disease. Specifically, such a method can comprise the steps of:

(a) providing a test sample (which can optionally be stored in a sample collection tube containing a MPO secretion inhibitor such as that described in Section D);

(b) determining the concentration of MPO in the test sample according to any of the assays described herein (e.g., using the methods described in this Section E, namely, methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies); and

(c) comparing the concentration of MPO in the test sample determined in step (b) with a predetermined level. Specifically, when making such a comparison, if the concentration of MPO determined in step (b) is lower than the predetermined level, then the subject is considered not to be at risk of developing cardiovascular disease. However, when making such a comparision, if the concentration of MPO in the test sample determined in step (b) is higher then the predetermined level, then the subject is considered to be at risk of developing cardiovascular disease.

The concentration of MPO in a test sample determined using the methods described herein can also be useful to provide an indicator of the clinical status (i.e., severity or progression of disease) of a subject. For example, the concentration of MPO determined using the methods of the present disclosure can be used to determine whether or not subject is suffering from a disease such as heart failure. Alternatively, the concentration of MPO determined as described herein can be used to determine whether a subject suffering from heart failure should be classified in any of New York Heart Association (NYHA) Classifications I, II, III or IV or whether a subject classified as certain New York Heart Association Classification has progressed to a different New York Heart Association Classification (e.g., the subject was initially classified as New York Heart Association Classification II and then the subject progress to New York Heart Association Classification III). Specifically, the severity or progression of disease, such as cardiovascular disease, in a subject can be determined using a method comprising the steps of:

(a) providing a test sample from a subject (which can optionally be stored in a sample collection tube containing a MPO secretion inhibitor such as that described in Section D);

(b) determining the concentration of MPO in the test sample according to any of the assays described herein (e.g., using the methods described in this Section E, namely, methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies);

(c) comparing the concentration of MPO in the test sample determined in step (b) with a predetermined level. Specifically, when making such a comparison with respect to severity of cardiovascular disease in a subject, if the concentration of MPO determined in step (b) is lower than the predetermined level, the subject is determined to have a reduced severity of cardiovascular disease. If the concentration of MPO determined in step (b) is higher than the predetermined level, the subject is determined to have an increased severity of cardiovascular disease. When comparing the concentration of MPO in the test sample determined in step (b) with respect to progression of cardiovascular disease, if the concentration of MPO determined in step (b) is lower or unchanged to a predetermined level, the subject is determined not to have progressed or to have improved with respect to cardiovascular disease. If the concentration of MPO in the test sample determined in step (b) is higher when compared to a predetermined level, the subject is determined to have progressed with respect to cardiovascular disease. The progression of disease, such as cardiovascular disease, can be monitored either before treatment is commenced in a subject or after commencement of treatment in a subject.

Moreover, the concentration of MPO determined using the methods described herein can be used to determine if a subject has suffered a cardiovascular complication as a result of administration to said subject of one or more pharmaceutical compositions. For example, such a method can comprise the steps of:

(a) obtaining a first test sample from the subject before the subject has been administered one or more pharmaceutical compositions (which can optionally be stored in a sample collection tube containing a MPO secretion inhibitor such as that described in Section D);

(b) determining the concentration of MPO in the test sample according to any of the assays described herein (e.g., using the methods described in this Section E, namely, methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies);

(c) obtaining a second test sample from the subject after the subject has been administered one or more pharmaceutical compositions (which can optionally be stored in a sample collection tube containing a MPO secretion inhibitor such as that described in Section D);

(d) determining the concentration of MPO in the second test sample according to any of the assays described herein (e.g., using the methods described in this Section E, namely, methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies); and (e) comparing the concentration of MPO in step (b) with the concentration of MPO in step (d). Specifically, if the concentration of MPO determined in step (b) is unchanged when compared to the concentration of MPO determined in step (d), then the subject is determined not to have suffered a cardiovascular complication as a result of the administration of one or more pharmaceutical compositions. Moreover, if the concentration of MPO determined in step (b) is changed when compared to the concentration of MPO in step (d), then the subject is determined to have suffered a cardiovascular complication as a result of the administration of one or more pharmaceutical compositions.

Still further, the concentration of MPO determined using the methods described herein can be used in methods for monitoring MPO levels in a subject receiving treatment with one or more pharmaceutical compositions. Specifically, such methods involve providing a first test sample from a subject before the subject has been administered one or more pharmaceutical compositions (which can optionally be stored in a sample collection tube containing a MPO secretion inhibitor such as that described in Section D). Next, the concentration of MPO (e.g., the level of MPO) in the test sample is determined according to any of the assays described herein (e.g., using the methods described in this Section E, namely, methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies). After the concentration of MPO in the test sample is determined, the concentration of MPO is then compared with a predetermined level. If the concentration of MPO determined in the first test sample is lower then the predetermined level, then the subject is not treated with one or more pharmaceutical compositions. However, if the concentration of MPO determined in the first test sample is higher then the predetermined level, then the subject is treated with one or more pharmaceutical compositions for a period of time. The period of time that the subject is treated with the one or more pharmaceutical compositions can be determined by one skilled in the art (for example, the period of time can be from about seven (7) days to about two years, preferably from about fourteen (14) days to about one (1) year). During the course of treatment with the one or more pharmaceutical compositions, second and subsequent test samples are then obtained from the subject (any or all of these test samples can optionally be stored in a sample collection tube containing a MPO secretion inhibitor such as that described in Section D). The number of test samples and the time in which said test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second and subsequent test sample is obtained from the subject, the concentration of MPO in the second and subsequent test samples is determined according to any of the assays described herein (e.g., using the methods described in this Section E, namely, methods that use an antibody produced by murine hybridoma cell line 1-1175-509 having A.T.C.C. Accession No. PTA-8437, an antibody produced by murine hybridoma cell line 1-2169-715 having A.T.C.C. Accession No. PTA-8438 or both of these antibodies). The concentrations of MPO determined in each of these second and subsequent test samples is then compared with the concentration of MPO determined in the first test sample (e.g., the test sample that was originally compared to the predetermined level). If the concentrations of MPO determined in the second and subsequent test samples are lower or have decreased when compared to the concentration of MPO determined in the first test sample, then treatment with the one or more pharmaceutical compositions can be continued. However, if the concentrations of MPO determined in the second and subsequent test samples are the same or have increased when compared to the concentration of MPO determined in the first test sample, then a determination is made that the one or more pharmaceutical composition are not efficacious for reducing MPO levels in that subject. The subject can then is either: (a) treated with a higher dose of the one or more pharmaceutical compositions when compared to the dose of the one or more pharmaceutical compositions previously given to said subject; or (b) switched to one or more alternate or different pharmaceutical compositions. Specifically, the subject can be treated with one or more pharmaceutical compositions that are different then the one or more pharmaceutical compositions that the subject had previously received to decrease or lower said subject's MPO levels.

### F. MPO Kits

The present disclosure also contemplates kits for detecting the presence of MPO in a test sample. Such kits can comprise one or more of the antibodies described herein. More specifically, if the kit is a kit for performing an immunoassay, the kit optionally can contain (1) at least one capture antibody that specifically binds to MPO; (2) at least one conjugate; and (3) one or more instructions for performing the immunoassay. The antibodies of the present disclosure can be included in such a test kit as a capture antibody, as a detection antibody or both as a capture antibody and a detection antibody. For example, an antibody produced by murine hybridoma cell line 1-1175-509 can be included in the kit as capture antibody and an antibody produced by murine hybridoma cell line 1-2169-715 can be included in the kit as a detection antibody. Alternatively, an antibody produced by murine hybridoma cell line 1-2169-715 can be included in the kit as a capture antibody and an antibody produced by hybridoma cell line 1-1175-509 can be included in the kit as a detection antibody. In still yet another alternative, an antibody produced by murine hybridoma cell line 1-1175-509 or an antibody produced by murine hybridoma cell line 1-2169-715 can be included in the kit as a capture antibody and a different antibody included in the kit as a detection antibody. In still yet another alternative, an antibody produced by murine hybridoma cell line 1-1175-509 or an antibody produced by murine hybridoma cell line 1-2169-715 can be included in the kit as a detection antibody and a different antibody included in the kit as a capture antibody. Optionally, the kit can also contain at least one calibrator or control. Any calibrator or control can be included in the kit. Preferably, however, the calibrator or control is a purified MPO. Optionally, the kit can also contain at least one sample collection tube. Optionally, the kit can also contain at least one leukocyte MPO secretion inhibitor. An example of at least one leukocyte MPO secretion inhibitor that can be included in the kit is a salt of any salt of EDTA or sodium citrate. Alternatively, the kit can also containing at least one sample collection tube that contains at least one leukocyte MPO secretion inhibitor.

Thus, the present disclosure further provides for diagnostic and quality control kits comprising one or more antibodies of the present disclosure. Optionally the assays, kits and kit components of the invention are optimized for use on commercial platforms (e.g., immunoassays on the PRISM®, AxSYM®, ARCHITECT® and EIA (Bead) platforms of Abbott Laboratories, Abbott Park, Ill., as well as other commercial and/or in vitro diagnostic assays). Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories, Abbott Park, Ill.) electrochemical immunoassay system that performs sandwich immunoassays for several cardiac markers, including TnI, CKMB and BNP. Immunosensors and methods of operating them in single-use test devices are described, for example, in U.S. Patent Applications 20030170881, 20040018577, 20050054078 and 20060160164 which are incorporated herein by reference. Additional background on the manufacture of electrochemical and other types of immunosensors is found in U.S. Pat. No. 5,063,081 which is also incorporated by reference for its teachings regarding same.

Optionally the kits include quality control reagents (for example, sensitivity panels, calibrators, and positive controls). Preparation of quality control reagents is well known in the art, and is described, e.g., on a variety of immunodiagnostic product insert sheets.

In another embodiment, the present disclosure provides for a quality control kit comprising one or more antibodies of the present disclosure for use as a sensitivity panel to evaluate assay performance characteristics and/or to quantitate and monitor the integrity of the antigen(s) used in the assay.

The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

The kit further can optionally include instructions for use, which may be provided in paper form or in computer-readable form, such as a disc, CD, DVD or the like.

### G. Adaptations of the Methods

The disclosure herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM®, IMx®, PRISM®, and Quantum™ II instruments, as well as other platforms. Moreover, the disclosure optionally is adaptable for the Abbott Laboratories commercial Point of Care (i-STAT™) electrochemical immunoassay system for performing sandwich immunoassays. Immunosensors, and their methods of manufacture and operation in single-use test devices are described, for example in, U.S. Pat. No. 5,063,081, U.S. Patent Application 2003/0170881, U.S. Patent Application 2004/0018577, U.S. Patent Application 2005/0054078, and U.S. Patent Application 2006/0160164, which are incorporated in their entireties by reference for their teachings regarding same.

By way of example, and not of limitation, examples of the present disclosure shall now be given.

### EXAMPLE 1

### A.T.C.C. Deposit Information

Murine hybridoma cell line for monoclonal antibody 1-1175-509 was deposited with the American Type Culture Collection (hereinafter referred to as “A.T.C.C.”), 10801 University Blvd., Manassas, Va. 20110-2209, on May 16, 2007 and assigned A.T.C.C. Accession No. PTA-8437.

Murine hybridoma cell line for monoclonal antibody 1-2169-715 was deposited with the A.T.C.C., 10801 University Blvd., Manassas, Va. 20110-2209, on May 16, 2007 and assigned A.T.C.C. Accession No. PTA-8438.

### EXAMPLE 2

### Evaluation of Specimen Collection Tubes

The effect of specimen collection tube anticoagulant or lack of coagulant and glass vs. plastic tube were studied. Blood samples from 15 normal donors collected in 9 different sample tube types were purchased from a commercial vendor. Samples were centrifuged, plasma/serum separated, stored at 2-8° C. and shipped to the processing site. The MPO values were measured using an experimental sandwich immunoassay performed on the ARCHITECT® automated immunoassay instrument (sold by Abbott Laboratories, Abbott Park, Ill.). The experimental ARCHITECT® assay is described in S. Datwyler et al., “Development of an Automated Myeloperoxidase (MPO) Immunoassay”, Clinical Chemistry. 52. No. 6. Supplement; D-11, 2006. The analytical range of the assay is from 5 to 10,000 pmol/L with precision ranging from 2.4 to 6.0% CV over the range of 250 to 5000 pmol/L. The two monoclonal antibodies used in the experimental ARCHITECT® assay are 1-1175-509 and 1-2169-715.

The sample tube types and measured mean MPO values are in the following Table 1:

TABLE 1

| | | Mean MPO |
|---|---|---|
| Anticoagulant | Type Type | (pmol/L) |
| Lithium Heparin | Plastic | 100.9 |
| PST (Lithium heparin | Plastic | 101.2 |
| plasma separator tube) | | |
| Sodium Heparin | Plastic | 99.5 |
| Lithium Heparin | Glass | 109.6 |
| Serum | Glass | 144.7 |
| Serum | Plastic | 191.6 |
| SST (Serum Separator tube) | Plastic | 149.3 |
| EDTA | Plastic | 91.9 |
| Citrate | Plastic | 93.7 |

The lowest concentration of MPO was found with EDTA and citrate plasma samples. The samples collected in heparin storage tubes showed mean MPO levels approximately 10% higher in value than the preferred EDTA tube. Serum samples were markedly higher in mean MPO level. This data supports that the conventional use of lithium heparin plasma storage conditions and serum for MPO measurements overmeasures MPO levels.

### EXAMPLE 3

### Centrifugation Studies

Samples were collected from six healthy donors to evaluate the effect of sample handling on MPO values. Both plastic lithium heparin plasma (4 mL, BD 367884) tubes and lithium heparin plasma separator tubes (PST) (4.5 mL, BD 367962) purchased from BD, were evaluated for the effect of centrifugation speed, clotting time and storage temperature.

The first study evaluated the effect of centrifugation speed and duration. Samples were immediately placed at 2-8° C. after completion of collection from each donor and then centrifuged as shown in Table 2:

TABLE 2

| Tube | | |
|---|---|---|
| Number | Time | Speed |
| 1 | 5 min | 1000 × g |
| 2 | 10 min | 500 × g |
| 3 | 5 min | 500 × g |
| 4 | 10 min | 1250 × g |

Samples were then tested immediately with the ARCHITECT® assay used in Example 2. MPO concentrations were not impacted by centrifugation time or speed when samples are placed at 2-8° C., centrifuged and immediately assayed for MPO. Lithium heparin plasma tubes and PST tubes provided equivalent results.

The next study evaluated the effect of temperature on storage of the sample prior to centrifugation. Upon sample collection tubes were placed at 2-8° C. until samples from all donors were collected. Then tubes were placed either at room temperature or at 2-8° C. prior to centrifugation. Samples were then centrifuged after 30 minutes, 2 hours or 6 hours. Centrifugation was for 10 minutes at 1250 g. Samples were then tested immediately with the ARCHITECT® assay used in Example 2. The results are shown in Table 3, including the mean MPO values of the 6 donors at each timepoint and storage condition. Samples in lithium heparin plasma and PST tubes were stable for 6 hours at 2-8° C.; however samples in tubes stored at room temperature were unstable and demonstrated a marked increase in myeloperoxidase over time. These data show the potential for significant change in MPO levels due to room temperature storage when a leukocyte MPO secretion inhibitor is not used.

TABLE 3

| Storage | | Li Heparin | Li Heparin PST |
|---|---|---|---|
| Condition | Time | (pmol/L) | (pmol/L) |
| | Baseline | 137 | 141 |
| 2-8° C. | 0.5 hrs | 122 | 111 |
| | 2 hrs | 125 | 96 |
| | 6 hrs | 140 | 116 |
| Room Temp | 0.5 hrs | 152 | 140 |
| | 2 hrs | 286 | 332 |
| | 6 hrs | 913 | 1480 |

### EXAMPLE 4

### Investigation of Additional Tube Types for Sample Stability

Samples were collected from 5 healthy donors to further examine the effect of collection tube type and handling on MPO determinations. Lithium heparin (4 mL, BD 367884), K₂EDTA (4 mL, BD367862), K₂EDTA plasma separator tube (PPT)(5 mL, BD362788) and sodium citrate (2.7 mL, BD363083) collection tubes were purchased from Becton Dickinson and Company (Franklin Lakes, N.J.). After collection, samples were stored at either room temperature (15-30° C.) or at (2-8° C.), centrifuged at 0, 2, and 8 hours (10 minutes, 1250 g), plasma removed from the cells and tested using the ARCHITECT® assay of Example 2 for MPO. EDTA plasma, in either regular EDTA or PPT tubes, was the most stable at both room temperature and 2-8° C. The results shown in Table 4 are the mean MPO values of the 5 donors at each timepoint and storage condition. Citrate and lithium heparin storage tubes were able to maintain MPO levels relatively stable with storage at 2-8° C. for 2 hours, note however the MPO concentration in citrate was similar to the concentration measured in EDTA, whereas the concentration in lithium heparin was increased over the concentration in EDTA or citrate at baseline. Storage at room temperature for both citrate and lithium heparin showed marked increase in MPO levels over time. In contrast, use of the storage tubes with the leukocyte MPO secretion inhibitor EDTA was able to maintain MPO levels essentially constant at both room temperature and 2-8° C.

TABLE 4

| Storage | | EDTA | EDTA PPT | Li Heparin | Citrate |
|---|---|---|---|---|---|
| Condition | Time | (pmol/L) | (pmol/L) | (pmol/L) | (pmol/L) |
| | Baseline | 91 | 87 | 130 | 83 |
| 2-8 | 2 hrs | 86 | 88 | 112 | 95 |
| degrees C. | 8 hrs | 99 | 99 | 159 | 100 |
| Room | 2 hrs | 102 | 93 | 465 | 432 |
| Temp | 8 hrs | 112 | 103 | 992 | 1615 |

### EXAMPLE 5

### Stability of Separated Plasma Samples

Samples from 10 normal donors were collected in Lithium Heparin PST (8 mL, BD367964), SST (8.5 mL, BD367988) and EDTA PPT (5 mL, BD362788) tube types. PST and PPT tubes were centrifuged immediately; SST tubes were allowed to clot for 30 minutes at room temperature prior to centrifugation. The isolated plasma or serum were tested neat and spiked with MPO. Samples were then placed at room temperature and tested at time 0, 3.5 hours, 24 hours and 48 hours. Samples were also placed at 2-8° C. and tested using the ARCHITECT® assay of Example 2 at time 0, 24 hours, 48 hours, 5 days and 8 days. As seen in Tables 5 and 6 the isolated plasma or serum was stable under all conditions tested.

TABLE 5
Room Temperature Stability

| | | Percent Change | | |
|---|---|---|---|---|
| | | from Time 0 | | |
| Tube Type | N | 3.5 hr | 24 hr | 48 hr |
| PST | 13 | −2.5 | −2.9 | −1.7 |
| SST | 10 | −1.3 | −3.6 | −6.5 |
| PPT | 10 | 0.1 | −0.6 | −0.1 |

TABLE 6
2-8° C. Stability

Percent Change from Time 0

| Tube Type | N | Day 1 | Day 2 | Day 5 | Day 8 |
|---|---|---|---|---|---|
| PST | 13 | −3.9 | −2.7 | −3.3 | −7.0 |
| SST | 10 | −2.4 | −2.1 | −2.5 | −6.8 |
| PPT | 10 | 0.4 | 1.4 | 0.1 | −3.2 |

### EXAMPLE 6

### Spike Recovery Studies

To further investigate the effect of anticoagulant on MPO detection, spike recovery experiments were performed to determine if the presence of anticoagulant in the plasma affected the determination of MPO. Twenty-five matched sets were purchased from BioCollections Worldwide, Inc. (Miami, Fla.). Samples were collected into lithium heparin, K₂EDTA and sodium citrate tubes, centrifuged and shipped at 2-8° C. Each sample was >5 mL. Each sample was split into three aliquots. One set was spiked with 300 pmol/L native MPO antigen (Athens Research and Technology, Athens, Ga.) and a second set spiked with 3000 pmol/L MPO antigen. The neat and spiked samples were run on the ARCHITECT® assay of Example 2 and the Grand Mean percent Difference calculated for each tube type. As seen in Table 7, there was no effect by either the EDTA or citrate storage tubes on MPO recovery; lithium heparin showed a small effect.

TABLE 7

| | % Recovery | % Recovery | |
|---|---|---|---|
| | 300 pmol/L | 3000 pmol/L | % Grand Mean |
| Tube Type | spike | spike | Recovery |
| Heparin | 88 | 94 | 91 |
| EDTA | 102 | 96 | 99 |
| Citrate | 99 | 96 | 98 |

### EXAMPLE 7

### Testing Naturally Elevated Samples in Various Tube Types

Fifty matched specimens were collected from patients presenting to the Emergency Department with chest pain and/or other complaints leading to clinically indicated blood draws. The collection tubes evaluated were plastic serum (SST), K₂EDTA plasma, lithium heparin plasma (PST) and citrate tubes. All samples were stored at room temperature for no more than 60 minutes prior to centrifugation except the EDTA samples that were stored at 2-8° C. as whole blood samples for 24 hours prior to centrifugation. Separated serum and plasma were stored at 2-8° C. until tested by the ARCHITECT® MPO assay of Example 2. The mean, median, minimum and maximum concentration for each tube type are summarized in the Table 8:

TABLE 8

| | | Mean | Median | Minimum | Maximum |
|---|---|---|---|---|---|
| Tube Type | N | (pmol/L) | (pmol/L) | (pmol/L) | (pmol/L) |
| Serum | 50 | 917.7 | 643.5 | 188.0 | 4882.0 |
| EDTA | 50 | 313.6 | 149.0 | 31.0 | 3791.0 |
| Heparin | 50 | 840.6 | 493.0 | 122.0 | 8252.0 |
| Citrate | 50 | 300.7 | 127.8 | 44.4 | 3501.1 |

These results compare favorably with the results from Example 2. The lowest values are found with EDTA and citrate samples. For most of the samples, the serum sample provided the highest MPO values.

### EXAMPLE 8

### Animal Immunizations

RBF/DnJ female mice (The Jackson Laboratory, Bar Harbor, Me.) were immunized three times with purified MPO antigen (Advanced Immunochemical, Long Beach, Calif.), using either the Freund's Adjuvant (Difco, Detroit, Mich.) or Ribi MPL+TDM (Corixa, Hamilton, Mont.) Adjuvant System. Mouse numbers 1926 and 1930 received the Freund's system and mouse numbers 1932, 1933, and 1935 received Ribi system. The inoculum was prepared by diluting the MPO antigen in 0.9% sodium chloride (Abbott Laboratories, Abbott Park, Ill.) and emulsifying with one of the two adjuvants. At weeks 0, 13, and 21, a 10 μg boost of MPO was administered to the mice. Freund's Complete Adjuvant was used for the primary boost and delivered subcutaneously. Freund's Incomplete Adjuvant was used for the next 2 boosts delivered through intradermal injection. Ribi Adjuvant was used for all three immunizations on the Ribi mice, and each was delivered by intradermal injection. Three days prior to the fusion, the mice were administered a pre-fusion boost of 20 μg of MPO.

### EXAMPLE 9

### Development of murine MPO mAbs

On the day of fusion, the mice were euthanized and their spleens containing anti-MPO splenocytes were harvested and placed into Iscove's Modified Dulbecco's Medium (IMDM) (Invitrogen Corporation, Grand Island N.Y.). A cell fusion was performed as described by Kohler and Milstein (Nature, 256:495-7 (1975)). Each mouse spleen was placed into a separate petri dish containing IMDM. The splenocytes were perfused out of each spleen using a syringe containing IMDM and cell scraper, then counted using a hemocytometer. Approximately 5.0×10⁶ splenocytes were pooled from each mouse and washed by centrifugation into a cell pellet and re-suspended in IMDM. These splenocytes were mixed with an equal number of SP 2/0 myeloma cells and centrifuged into a pellet. The fusion was accomplished by exposing the splenocytes and SP 2/0 cells to 50% polyethylene glycol (PEG) (A.T.C.C. Molecular Weight 1300-1600, Manassas Va.) in IMDM. Two mL of the PEG solution was added to the cell pellet followed by a one-minute incubation. The PEG and cell pellet was diluted by slowly adding thirty mL of IMDM over 30 seconds. The fused cells were then removed from suspension by centrifugation and decanting the supernatant. The cell pellet was re-suspended into 502 mL containing an approximate 50% mixture of spent medium from the SP 2/0 myeloma cell culture and fresh IMDM supplemented with FBS (Hyclone Laboratories, Logan Utah), HAT (Hypoxanthine, Aminopterin, Thymidine) (Sigma Laboratories, St. Louis, Mo.), Hybridoma Cloning Factor (Bioveris Corporation, Gaithersburg Md.), and L-Glutamine (Invitrogen Corporation, Grand Island N.Y.) in order to select for hybridomas. The cells were plated at 0.2 mL per well into twenty-five 96 well cell culture plates. After incubating from 3-5 days one half of the medium in each well was removed by aspiration and replaced with IMDM supplemented with 10% FBS, HT Supplement, and L-glutamine. This procedure was completed twice and the hybridomas were allowed to grow for 7-10 days prior to supernatant screening for antibody production.

Cell supernatant samples were analyzed for anti-MPO antibodies by EIA. Goat anti-mouse IgG Fc (Jackson Immunoresearch, West Grove Pa.) was coated on 96 well microtiter EIA plates at 5 ug/mL. After the capture reagent has been coated on the solid phase, it was removed and any open binding sites on the plates were blocked using BSA or Fish Gelatin block solution. Cell supernatants were then added to the blocked plates and allowed to incubate at room temperature for at least one hour. The anti-mouse IgG Fc will capture the anti-MPO mouse antibody from the supernatant. Following the incubation, the supernatants were washed off using distilled water. MPO antigen was added to the plates at 500 ng/mL and incubated for approximately 30 minutes. Following this incubation, the antigen was washed from the plates using distilled water. Rabbit anti-MPO antibody was added to the plates at 250 ng/mL and incubated for approximately 30 minutes. Following this incubation, the antibody was washed from the plates using distilled water. Goat anti-rabbit-HRPO (Kirkegaard & Perry) diluted to approximately 250 ng/mL in block solution was added to the plates and allowed to incubate for 30 minutes. The plates were washed with distilled water to remove the Goat anti-rabbit-HRPO and o-phenylenediamine substrate (OPD; Abbott Laboratories, Abbott Park, Ill.) was used as the chromogen to generate signal. Plates were read at 492 nm and the results were analyzed. Hybrids were considered positive if they had an EIA signal at least 3 times greater than background (See Table 9, below).

TABLE 9

| Hybrid No. | Background | PC | Hybrid |
|---|---|---|---|
| 1-1175 | 0.1 | 0.26 | 0.54 |
| 1-2169 | 0.1 | 0.37 | 0.54 |

Positive hybrids were expanded to 24 well plates in IMDM supplemented with 10% FBS and HT supplement. Following 3-7 days growth, the 24 well cultures were evaluated by EIA as described in this example, except the antibody supernatant was tittered to look for a dose response (See, Table 10, below).

TABLE 10

| Hybrid No. | Dilution | BSA Blank | MPO |
|---|---|---|---|
| 1-1175 | 1:64 | 0.08 | 0.62 |
| 1-2169 | 1:64 | 0.07 | 0.43 |
| Ms # 1922 | 1:3200 | 0.08 | 0.46 |
| serum | | | |

Hybrid MPO binding epitope groups were determined by measuring the ability of each MPO hybrid mAb to complete a mAb-antigen-mAb sandwich with five existing outside vendor monoclonal antibodies (See, Table 11, below) that are commercially available and were biotin labeled and believed to have different and distinct binding epitopes on MPO. Briefly, rabbit anti-mouse IgG Fc (Jackson Immunoresearch, West Grove, Pa.) was coated on 96 well microtiter EIA plates at 10 ug/mL. After the capture reagent has been coated on the solid phase, it was removed and any open binding sites on the plates were blocked using BSA or Fish Gelatin block solution. Cell supernatants were then added to the blocked plates and allowed to incubate at room temperature for at least one hour. The anti-mouse IgG Fc will capture the anti-MPO mouse antibody from the supernatant. Following the incubation, the supernatants were washed off using distilled water and any unbound rabbit anti-mouse IgG binding sites were blocked with normal mouse serum diluted in block solution. After that was washed off, MPO antigen was added to the plates at 400 ng/mL and incubated for approximately 60 minutes. Following the incubation, the antigen was washed from the plates using distilled water. The biotin labeled outside vendor mouse anti-MPO antibodies were added to the plates and incubated for approximately 30 minutes. Following this incubation, the antibody was washed from the plates using distilled water. Streptavidin-HRPO diluted to approximately 100 ng/mL in block solution was added to the plates and allowed to incubate for 30 minutes. The plates were washed and o-phenylenediamine substrate was used as the chromogen to generate signal. Plates were read at 492 nm and the results were analyzed. The supernatant dilution that generates approximately 50% of maximal binding was compared for each of the hybrids. Table 11, below, summarizes the absorbance values for each hybrid supernatant with each of the biotin labeled mAbs. Based on their ability to form sandwiches with each of these antibodies, the hybrids were divided into groups. Hybrids 1-1175 and 1-2169 were believed to bind to distinct epitopes on MPO and were therefore selected for cloning to stabilize the cell line and ensure there that there was no mixed cell population.

TABLE 11

| | | | | | | M01102821- | M01102822- |
|---|---|---|---|---|---|---|---|
| Hybrid # | Dilution | Blank | 2A11-Bt | 3B1-Bt | 4B2-Bt | Bt | Bt |
| 1175 | 1:64 | 0.12 | 1.40 | 1.86 | 0.63 | 0.62 | 1.05 |
| 2244 | 1:64 | 0.09 | 0.67 | 0.69 | 0.74 | 0.91 | 0.48 |
| 1519 | 1:64 | 0.16 | 0.33 | 1.35 | 1.12 | 1.50 | 0.64 |
| 2169 | 1:64 | 0.10 | 0.67 | 0.92 | 0.76 | 1.06 | 0.60 |

Hybrids 1-1175 and 1-2169 were cloned using a Fluorescent Activated Cell Sorter (FACS). Hybrid cultures were stained with Propidium Iodide to stain non-viable cells so they could be deselected from the population. The stained cultures were analyzed on the FACSAria (BD Biosciences). Single cells were deposited into each well of 96 well culture dishes containing Hybridoma Serum Free Medium (HSFM) supplemented with L-Glutamine and 10% FBS. The cultures were allowed to incubate for approximately 7 days and screened by EIA, as previously described in this example. Clones 1-1175-154 and 1-2169-143 were isolated and purified antibody was generated from each cell line.

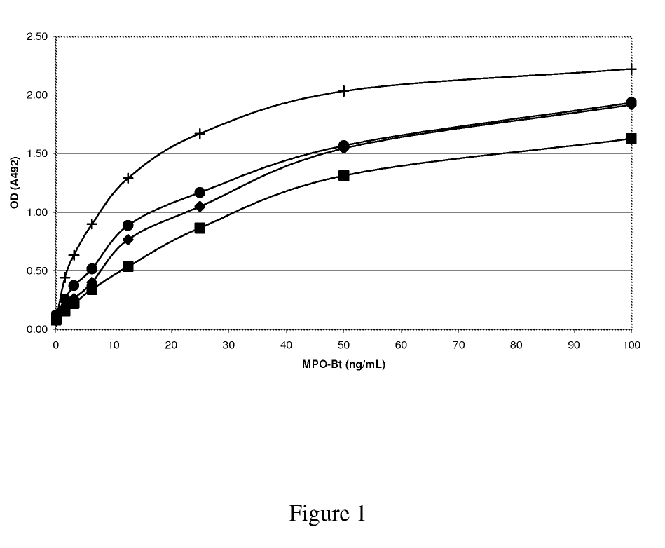

The purified antibody was tested for it's relative affinity ranking with the other antibodies generated (See, Table 11). Rabbit anti-mouse IgG Fc (Jackson Immunoresearch) was coated on 96 well microtiter EIA plates at 5 ug/mL. After the capture reagent has been coated on the solid phase, it was removed and any open binding sites on the plates were blocked using BSA or Fish Gelatin block solution. Purified antibody was added to the plates at six different dilutions and allowed to incubate for approximately 60 minutes. The antibody was washed off and biotin labeled MPO antigen was added in concentrations from 0 to 100 ng/mL and allowed to incubate for 60 minutes. The antigen was washed off and streptavidin-HRPO diluted to approximately 100 ng/mL in block solution was added to the plates and allowed to incubate for 30 minutes. The plates were washed and o-phenylenediamine substrate was used as the chromogen to generate signal. Plates were read at 492 nm and the results were analyzed. The antibody concentration generating approximately half of the maximum binding signal was used to generate an antigen titration curve shown in FIG. 1.

Purified antibody from each of these clones was labeled with biotin. Epitope group confirmation was completed using a competitive inhibition micro-titer assay, using a number of different proprietary and commercial available monoclonal antibodies. MPO antigen was coated on 96 well microtiter EIA plates at 0.5 ug/mL. After the capture reagent had been coated on the solid phase, it was removed and any open binding sites on the plates were blocked using BSA or Fish Gelatin block solution. Purified antibody (100 uL/well) from each clone at 50 ug/mL was then added to the blocked plates and allowed to incubate at room temperature for one hour. Biotin labeled antibody (50 uL/well) was then added to the wells, without washing out the unlabeled antibody, and allowed to incubate for approximately 10-15 minutes. After that incubation, the antibody solution was washed from the plate using distilled water. Streptavidin-HRPO diluted to approximately 200 ng/mL in block solution was added to the plates and allowed to incubate for 30 minutes. The plates were washed and o-phenylenediamine substrate was used as the chromogen to generate signal. Plates were read at 492 nm and the results were analyzed. The results indicated that unlabeled antibody from 1-1175-154 competes for binding to the MPO antigen with the labeled version of itself, but not the labeled version of 1-2169-143 (data not shown). The results also indicated that the unlabeled antibody from 1-2169-143 competes for binding to the MPO antigen with the labeled version of itself, but not the labeled version of 1-1175-154 (data not shown). These results demonstrate that these antibodies bind to distinctly different MPO epitopes and can form a mAb-Ag-mAb sandwich.

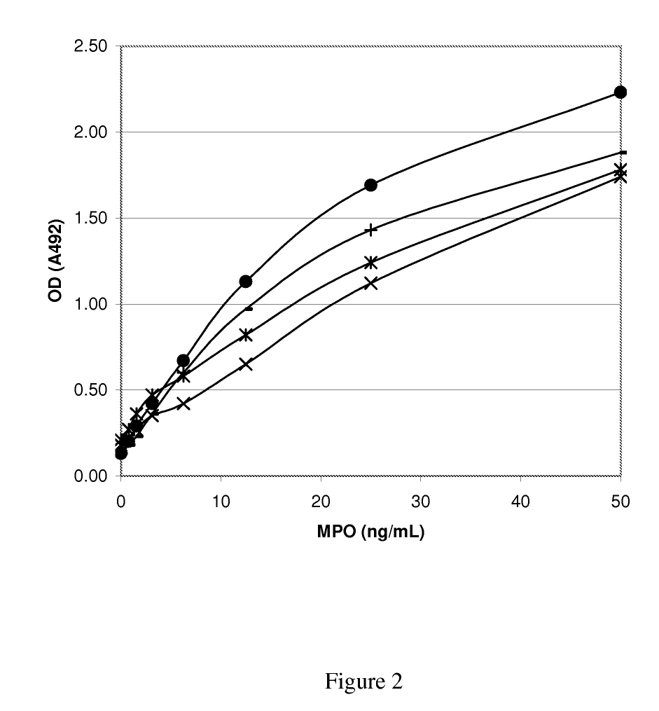

Antibody pairing studies confirmed the above data. Briefly, the unlabeled mAb is coated on to 96 well micro-titer plates at 1 ug/mL. After the capture reagent had been coated on the solid phase, it was removed and any open binding sites on the plates were blocked using BSA or Fish Gelatin block solution. MPO antigen from 0 to 50 ng/mL was then added to the blocked plates and allowed to incubate at room temperature for 30 minutes. Following this incubation, the antigen was washed from the plates using distilled water. The biotin labeled anti-MPO antibodies were added to the plates and incubated for approximately 30 minutes. Following this incubation, the antibody was washed from the plates using distilled water. Streptavidin-HRPO diluted to approximately 200 ng/mL in block solution was added to the plates and allowed to incubate for 30 minutes. The plates were washed and o-phenylenediamine substrate was used as the chromogen to generate signal. Plates were read at 492 nm and the results were analyzed. As shown in FIG. 2, these results confirmed that clones 1-1175-154 and 1-2169-143 were good MPO binding partners.

Clone 1-1175-154 was weaned to HSFM without FBS and subcloned using the FACS cell sorting method previously described. Cell line 1-1175-509 was selected for scale up and cell banking purposes. Liquid nitrogen freezers are used for long-term storage of the cell bank. Anti-MPO mAb 1-1175-154 is the parental clone from which subclone 1-1175-509 was derived.

Clone 1-2169-143 was weaned to HSFM without FBS and subcloned by counting the viable cells in culture and seeding 1 cell per well in 96 well tissue culture plates. The plates were allowed to incubate for 7-10 days and the subclone supernatant was tested for anti-MPO antibody using a micro-titer EIA. Cell line 1-2169-715 was selected for scale up and cell banking purposes. Liquid nitrogen freezers are used for long-term storage of the cell bank. Anti-MPO mAb 1-2169-143 is the parental clone from which subclone 1-2169-715 was derived.

The 1-1175-509 and 1-2169-715 cell lines were expanded in HSFM and seeded into roller bottles at approximately 0.5×10⁻⁵ cells/mL. The cultures were incubated at 37° C. while rotating at approximately 1 revolution per minute for 10-14 days, or until a terminal end culture was obtained. The terminal roller bottle supernatant was harvested and clarified with a 0.45 μM filter. The clarified supernatant was concentrated using a Pellicon system and filtered with a 0.45 mM filter. The mAb concentrate was diluted with an equal volume of 1.5 M glycine/3 N NaCl buffer at pH 8.9, then loaded onto a pre-equilibrated 5 ml Protein A column using the AKTA automated purification system (Amersham/Pharmacia). The column was then washed with 5 column volumes of binding buffer and when a stable baseline was achieved, the mAb was eluted with a pH 3.0 citrate buffer. The mAb was then transferred to a 70 mL G25 column for an exchange into PBS. The antibody was aliquoted and stored at −70° C.

Purified antibody from each of the 1-1175-509 and 1-2169-715 cell lines was tested with the Isostrip Mouse Monoclonal Antibody Isotyping Kit (Roche Diagnostics, Base1, Switzerland). An aliquot of 150 μL of 0.5 μg/mL to 3.0 ug/mL for each sample was added to the development tube and mixed. An Isostrip was added to each tube and incubated for 5-10 minutes until color development on the strip's band. The results indicated that 1-1175-509 is mouse IgG2b subtype with kappa light chain and 1-2169-715 is mouse IgG1 subtype with kappa light chain.

### EXAMPLE 10

### Affinity/Kinetic Characterization of Anti-MPO Antibody for MPO

The affinity of MPO monoclonal antibodies 1-1175-509 and 1-2169-143 for MPO was determined using a BIAcore 2000 instrument (BIAcore International AB, Uppsala, Sweden). First, a ˜5,000 RU goat anti-mouse IgG Fc Capture Biosensor was created by amine-coupling polyclonal goat anti-mouse IgG Fc antibody (Jackson Immunoresearch Laboratories) to a CM4 biosensor chip (BIAcore) via EDC/NHS/Ethanolamine chemistry provided in a Amine Coupling Kit (BIAcore). MPO antibody and MPO antigen were diluted into a running buffer (hereinafter “Running Buffer”) composed of HBS-EP buffer spiked with 0.1% BSA, 0.1% CM-Dextran, and 10 mM CaCl₂. Each MPO antibody was diluted to 0.6 μg/mL and MPO antigen (Athens Research & Technology, Athens, Ga.) was diluted to concentrations ranging from 0.0105 to 207 nM using a 3-fold dilution series.

After equilibrating the goat anti-mouse IgG Fc Capture Biosensor for 5 minutes at 10 μL/min with Running Buffer, 18 μL of MPO antibody was injected over individual flow cells with one flow cell being left blank as a reference flow cell. The flow cells were washed for 5 minutes at 75 μL/min with Running Buffer before injecting 150 μL of MPO antigen at a random concentration across the biosensor, which was immediately followed by 6 minutes of Running Buffer. The biosensor was regenerated with three 33 μL injections of 100 mM H₃PO₄ at a flow rate of 100 μL/minute. All concentrations of MPO antigen were tested in duplicate. The binding kinetics, association and dissociation, were monitored via sensorgrams. The sensorgrams were double-referenced and fit to a 1:1 binding model with mass transport using Scrubber 2.0 software (BioLogic Software Pty Ltd., Australia) to determine association and dissociation rates, as well as overall K_{D}. The results are shown in Table 12, below.

TABLE 12

| MPO | Epitope | kₒₙ | k_{off} | K_{D} |
|---|---|---|---|---|
| mAb | Group | (M⁻¹s⁻¹) | (s⁻¹) | (M) |
| 1-1175- | 3 | 1.60 (3) × 10⁸ | 9.8 (1) × 10⁻³ | 6.1 (1) × 10⁻¹¹ |
| 509 | | | | |
| 1-2169- | 5 | 9.24 (8) × 10⁶ | 7.22 (3) × 10⁻³ | 7.81 (3) × 10⁻¹⁰ |
| 143 | | | | |

Standard error of determined values is reported in parentheses with respect to the smallest number place value.

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A murine hybridoma cell line selected from the group consisting of: murine hybridoma cell line 1-1175-509 having A.T.C.C. Accession No. PTA-8437 and murine hybridoma cell line 1-2169-715 having A.T.C.C. Accession No. PTA-8438.

2. An antibody made from DNA extracted from a murine hybridoma cell line selected from the group consisting of: murine hybridoma cell line 1-1175-509 having A.T.C.C. Accession No. PTA-8437 and murine hybridoma cell line 1-2169-715 having A.T.C.C. Accession No. PTA-8438.

3. A monoclonal antibody produced by a murine hybridoma cell line selected from the group consisting of: murine hybridoma cell line 1-1175-509 having A.T.C.C. Accession No. PTA-8437 and murine hybridoma cell line 1-2169-715 having A.T.C.C. Accession No. PTA-8438.

4. An immunoassay for determining the concentration of myeloperoxidase (MPO) in a test sample, the immunoassay comprising the steps of:
(a) contacting a first capture antibody that binds to MPO with a test sample suspected of containing MPO to form a first capture antibody-MPO complex;
(b) contacting said test sample containing the first capture antibody-MPO complex with a second antibody that binds to MPO and that has been conjugated to a detectable label to form a second capture antibody-MPO-detection complex; and
(c) determining the amount of the capture antibody-MPO-detection complexes formed in step (b) by detecting the detectable label, wherein the amount of the second complexes formed is the amount of MPO contained in the test sample,
wherein either the first capture antibody or the second antibody is a monoclonal antibody selected from the group consisting of: a monoclonal antibody produced by murine hybridoma cell line 1-1175-509 having A.T.C.C. Accession No. PTA-8437 and a monoclonal antibody produced by murine hybridoma cell line 1-2169-715 having A.T.C.C. Accession No. PTA-8438

5. The immunoassay of claim 4, wherein the first capture antibody is immobilized on a solid phase to produce an immobilized antibody.

6. An immunoassay for determining the concentration of MPO in a test sample, the immunoassay comprising the steps of:
(a) contacting a first capture antibody that binds to MPO with a test sample suspected of containing MPO to form a first capture antibody-MPO complex, wherein the first capture antibody is an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438;
(b) contacting said test sample containing the first capture antibody-MPO complex with a second antibody that binds to MPO and that has been conjugated to a detectable label to form a second capture antibody-MPO-detection complex, wherein the second antibody is an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438, provided that the first capture antibody and the second antibody are not identical; and
(c) determining the amount of the capture antibody-MPO-detection complexes formed in step (b) by detecting the detectable label, wherein the amount of the second complexes formed is the amount of MPO contained in the test sample.

7. The immunoassay of claim 6, wherein the first capture antibody is immobilized on a solid phase to produce an immobilized antibody.

8. The immunoassay of claim 6, wherein the first capture antibody is an antibody produced by murine hybridoma cell line 1-1175-509 having A.T.C.C. Accession No. PTA-8437 and the second antibody is an antibody produced by murine hybridoma cell line 1-2169-715 having A.T.C.C. Accession No. PTA-8438.

9. The immunoassay of claim 8, wherein the first capture antibody is immobilized on a solid phase.

10. A kit comprising at least one antibody of claim 3 and instructions for using said kit.

11. A method for determining concentration of myeloperoxidase (MPO) in a test sample, the method comprising the steps of:
(a) providing a test sample stored in a sample collection tube containing a MPO secretion inhibitor; and
(b) determining the concentration of MPO in the test sample.

12. The method of claim 11, wherein the MPO secretion inhibitor comprises a salt of ethylene diamine tetraacetic acid.

13. The method of claim 11, wherein the test sample is a whole blood or plasma sample.

14. The method of claim 11, wherein step (b) is performed by a method selected from the group consisting of: a competitive immunoassay, a sandwich immunoassay, an enzyme-linked immunosorbent assay, an enzymatic assay and a clinical chemistry assay.

15. The method of claim 11, wherein the test sample is stored at room temperature for a period of time up to about 8 hours.

16. The method of claim 15, wherein the test sample is further stored at a temperature of from about 2° C. to about 8° C. for a period of up to about seven (7) days after processing.

17. The method of claim 11, wherein the MPO secretion inhibitor comprises a salt of citrate and the test sample is further stored at a temperature of from about 2° C. to about 8° C. for a period of up to about eight (8) hours prior to processing.

18. The method of claim 17, wherein the test sample is further stored at a temperature of from about 2° C. to about 8° C. for a period of up to about seven (7) days after processing.

19. An improved method for determining the concentration of MPO in a human blood sample, wherein said improvement comprises storing said sample in a sample collection tube containing a MPO secretion inhibitor at room temperature for a period of up to about 8 hours.

20. The method of claim 19, wherein the MPO secretion inhibitor comprises a salt of ethylene diamine tetraacetic acid.

21. The method of claim 19, wherein the blood sample is a whole blood.

22. The method of claim 19, wherein the concentration of MPO in the sample is performed by a method selected from the group consisting of: a competitive immunoassay, a sandwich immunoassay, an enzyme-linked immunosorbent assay, an enzymatic assay and a clinical chemistry assay.

23. The method of claim 19, wherein the sample is further stored at a temperature of from about 2° C. to about 8° C. for a period up of from up to about seven (7) days after processing.

24. An improved method for determining the concentration of MPO in a human blood sample, wherein said improvement comprises storing said sample in a sample collection tube containing a salt of citrate at a temperature from about 2° C. to about 8° C. for a period of up to about eight (8) hours prior to processing.

25. The method of claim 24, wherein the test sample is further stored at a temperature of from about 2° C. to about 8° C. for a period of up to about seven (7) days after processing.

26. A kit comprising:
(a) a sample collection tube containing a MPO secretion inhibitor;
(b) at least one antibody of claim 3; and
(c) instructions for using said kit.

27. An immunoassay for determining the concentration of MPO in a human peripheral blood sample, the immunoassay comprising the steps of:
(a) providing a human peripheral blood sample suspected of containing MPO stored in a sample collection tube containing a MPO secretion inhibitor;
(b) contacting a first capture antibody that binds to MPO with the human peripheral blood sample stored in a sample collection tube containing a MPO secretion inhibitor to form a first capture antibody-MPO complex, wherein the first capture antibody is an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438;
(c) contacting said human peripheral blood sample containing the first capture antibody-MPO complex with a second antibody that binds to MPO and that has been conjugated to a detectable label to form a second capture antibody-MPO-detection complex, wherein the second antibody is an antibody produced by murine hybridoma cell line 1-1175-509 having A.T.C.C. Accession No. PTA-8437 or an antibody produced by murine hybridoma cell line 1-2169-715 having A.T.C.C. Accession No. PTA-8438, provided that the first capture antibody and the second antibody are not identical; and
(d) determining the amount of the capture antibody-MPO-detection complexes formed in step (b) by detecting the detectable label, wherein the amount of the second complexes formed is the amount of MPO contained in the human peripheral blood sample.

28. The immunoassay of claim 27, wherein the first capture antibody is immobilized on a solid phase to produce an immobilized antibody.

29. The immunoassay of claim 27, wherein the first capture antibody is an antibody produced by murine hybridoma cell line 1-1175-509 having A.T.C.C. Accession No. PTA-8437 and the second antibody is an antibody produced by murine hybridoma cell line 1-2169-715 having A.T.C.C. Accession No. PTA-8438.

30. The immunoassay of claim 27, wherein the MPO secretion inhibitor comprises a salt of ethylene diamine tetraacetic acid.

31. The immunoassay of claim 27, wherein the test sample is a whole blood.

32. The immunoassay of claim 27, wherein the test sample is stored at room temperature for a period of time up to about 8 hours.

33. The immunoassay of claim 32, wherein the test sample is further stored at a temperature of from about 2° C. to about 8° C. for a period of up to about seven (7) days after processing.

34. The method of claim 27, wherein the MPO secretion inhibitor comprises a salt of citrate and the test sample is further stored at a temperature of from about 2° C. to about 8° C. for a period of up to about eight (8) hours prior to processing.

35. The method of claim 34, wherein the test sample is further stored at a temperature of from about 2° C. to about 8° C. for a period of up to about seven (7) days after processing.

36. A method of determining whether a subject is at risk of developing cardiovascular disease, the method comprising the steps of:
(a) providing a test sample stored in a sample collection tube containing a MPO secretion inhibitor; and
(b) determining the concentration of MPO in the test sample;
(c) comparing the concentration of MPO in the test sample determined in step (b) with a predetermined level, wherein if the concentration of MPO determined in step (b) is lower than the predetermined level, then the subject is considered not to be at risk of developing cardiovascular disease and further wherein, if the concentration of MPO in the test sample determined in step (b) is higher then the predetermined level, then the subject is considered to be at risk of developing cardiovascular disease.

37. The method of claim 36, wherein the concentration of MPO in the test sample in step (b) is determined pursuant to the method of claim 4.

38. The method of claim 36, wherein the concentration of MPO in the test sample in step (b) is determined pursuant to the method of claim 6.

39. A method of diagnosing cardiovascular disease in a subject, the method comprising the steps of:
(a) providing a test sample stored in a sample collection tube containing a MPO secretion inhibitor; and
(b) determining the concentration of MPO in the test sample;
(c) comparing the concentration of MPO in the test sample determined in step (b) with a predetermined level, wherein if the concentration of MPO determined in step (b) is lower than the predetermined level, then the subject is not be considered to have cardiovascular disease and further wherein, if the concentration of MPO in the test sample determined in step (b) is higher then the predetermined level, then the subject is considered to have cardiovascular disease.

40. The method of claim 39, wherein the concentration of MPO in the test sample in step (b) is determined pursuant to the method of claim 4.

41. The method of claim 39, wherein the concentration of MPO in the test sample in step (b) is determined pursuant to the method of claim 6.

42. A method of monitoring the severity of cardiovascular disease in a subject, the method comprising the steps of:
(a) providing a test sample stored in a sample collection tube containing a MPO secretion inhibitor;
(b) determining the concentration of MPO in the test sample; and
(c) comparing the concentration of MPO in the test sample determined in step (b) with a predetermined level, wherein if the concentration of MPO determined in step (b) is lower than the predetermined level, the subject is determined to have a reduced severity of cardiovascular disease and if the concentration of MPO in the test sample determined in step (b) is higher than the predetermined level, the subject is determined to have an increased severity of cardiovascular disease.

43. The method of claim 42, wherein the cardiovascular disease is coronary artery disease, peripheral vascular disease, hypertension, myocardial infarction or heart failure.

44. The method of claim 42, wherein the concentration of MPO in the test sample in step (b) is determined pursuant to the method of claim 4.

45. The method of claim 42, wherein the concentration of MPO in the test sample in step (b) is determined pursuant to the method of claim 6

46. A method of monitoring the progression of cardiovascular disease in a subject, the method comprising the steps of:
(a) providing a test sample stored in a sample collection tube containing a MPO secretion inhibitor;
(b) determining the concentration of MPO in the test sample; and
(c) comparing the concentration of MPO in the test sample determined in step (b) with a predetermined level, wherein if the concentration of MPO determined in step (b) is lower than the predetermined level, the cardiovascular disease in the subject is determined not to have progressed or to have improved and if the concentration of MPO in the test sample determined in step (b) is higher than the predetermined level, the cardiovascular disease in the subject is determined to have progressed.

47. The method of claim 46, wherein the concentration of MPO in the test sample in step (b) is determined pursuant to the method of claim 4.

48. The method of claim 46, wherein the concentration of MPO in the test sample in step (b) is determined pursuant to the method of claim 6.

49. A method of determining if a subject has suffered a cardiovascular complication as a result of administration to said subject of one or more pharmaceutical compositions, the method comprising the steps of:
(a) obtaining a first test sample from the subject before the subject has been administered one or more pharmaceutical compositions and storing said first test sample in a sample collection tube containing a MPO secretion inhibitor;
(b) determining the concentration of MPO in said sample;
(c) obtaining a second test sample from the subject after the subject has been administered one or more pharmaceutical compositions and storing said first test sample in a sample collection tube containing a MPO secretion inhibitor;
(d) determining the concentration of MPO in said second test sample; and
(e) comparing the concentration of MPO in step (b) with the concentration of MPO in step (d), wherein if the concentration of MPO determined in step (b) is unchanged when compared to the concentration of MPO determined in step (d), then the subject is determined not to have suffered a cardiovascular complication as a result of the administration of one or more pharmaceutical compositions and further wherein if the concentration of MPO determined in step (b) is changed when compared to the concentration of MPO in step (d), then the subject is determined to have suffered a cardiovascular complication as a result of the administration of one or more pharmaceutical compositions.

50. The method of claim 49, wherein the concentration of MPO in the test sample in step (b) or step (d) or both steps (b) and (d) is determined pursuant to the method of claim 4.

51. The method of claim 49, wherein the concentration of MPO in the test sample in step (b) or step (d) or both steps (b) and (d) is determined pursuant to the method of claim 6.

52. A method of monitoring MPO levels in a subject receiving treatment with one or more pharmaceutical compositions, the method comprising the steps of:
(a) providing a first test sample from the subject before the subject has been administered one or more pharmaceutical compositions, wherein said test sample is stored in a sample collection tube containing a MPO secretion inhibitor;
(b) determining the concentration of MPO in the first test sample;
(c) comparing the concentration of MPO determined in step (b) with a predetermined level;
(d) treating the subject with one or more pharmaceutical compositions for a period of time if the comparison of the concentration of MPO determined in step (c) is that the concentration of MPO in the first test sample is greater than the predetermined level;
(e) providing a second and subsequent test samples from the subject after the subject has been administered one or more pharmaceutical compositions, wherein said test samples are stored in a sample collection tube containing a MPO secretion inhibitor;
(f) determining the concentration of MPO in the second and subsequent test samples;
(g) compare the concentrations of MPO determined in step (f) with the concentration of MPO determined in step (b), wherein if the concentrations of MPO determined in step (f) decrease when compared to the concentration of MPO determined in step (b), then the subject should continue to be administered the one or pharmaceutical compositions of step (d), further wherein, if the concentrations of MPO determined in step (f) are the same or increase when compared to the concentration of MPO determined in step (b), then the subject should be treated with a higher concentration of the one or more pharmaceutical compositions administered to the subject in step (d) or the subject should be treated with one or more pharmaceutical compositions that are different then the one or more pharmaceutical compositions administered to the subject in step (d).

53. The method of claim 52, wherein the concentration of MPO in the test sample in step (b) or step (f) or both steps (b) and (f) is determined pursuant to the method of claim 4.

54. The method of claim 52, wherein the concentration of MPO in the test sample in step (b) or step (f) or both steps (b) and (f) is determined pursuant to the method of claim 6.


SEQUENCE LISTING (1 sequence)

SEQ ID NO: 1 - protein, 15 aa, Artificial Sequence
  Description of Artificial Sequence Synthetic peptide
GPAKELTPLKEAKVS